SEQUENCE LISTING (24 sequences)

SEQ ID NO: 1 - DNA, 333 bp, Zea Mays
acggcgcgtgggtgtaggatggcgagagctcgaacgagaagcgttggaggatggtgcatagcgtcatcttggcttccagcaacgcaaagctctggccgatgcagattctaggcccccctccgaacggaaagaaagcagcctgatgcctggttgcgctggagatgccgttggcaaacctttctgggttgaactcgcttgcgtcttttccccaaatgtcaggatcatggtgaatgaagatgacgggtagaaggaggtcaactcctgcaggatatttgattccaccgagctccatttccttataagttcttcttgttagaaaggttgccggtgggt

SEQ ID NO: 2 - DNA, 1833 bp, Zea mays
ggctgtatccgccatctgcttctctcttccactgccccaagccgccaccaatggcgacctgcgttctgctgatgctgcgggaagtctctccgtgggcgctggccagcgtggtggcgtccgtgtcgctgttgtggctggtggtctggacgctggagtgggcctggtggacgccttggcggctcgagcgggccctgcgggtccagggcctcaagggcaccaggtaccgcctcttcaccggcgacctgagggaaaccgcccgggctaaccgggaggctcgcaagaagccgctgccgctcggcagccacgacatcgccccacgcgtgcagcccatgcatcacagcaccatcaaggaatacgggaaactatcgttcacctggttcggcccaacaccaagggtgatgattcctgacccagagttagtcaaagaggtgctgtctaataagtttggccactttggcaaaccaaggagtaaccgcattgggaggttgctagccaacgggcttgtaaatcatgatggtgaaaaatgggcaaagcacaggagaattcttaatcctgcatttcaccatgaaaaaataaaggggatgatgccagtattttctacctgctgtattgaaatgattactagatgggataattcaatgccttctgagggatcttctgagatagatgtctggcctgagttccagaatcttactggagatgttatctcaagaactgcgtttgggagcaactatcaagaagggaggagaattttcgagctgcaaggagaactagctgaacgcctcatccaatctgttcagacaatatttatcccaggctattggttcttgcccaccaaaaacaacagaaggatgagagcgatcgatgtagagatccgcaaaattctccgtgaaataattgggaagagagagaaggatactaaaaacagagaaacaaataacgacgacttgctgggcttattactggagtcaaacacaaggcaatcaaatggaaatgcaagcctgggattgacaacagaagatgtgattgaggaatgcaagttattttactttgcaggtatggagacaacatcagtccttcttacttggacacttattgtgctaagcatgcacccagaatggcaagagagagcaagagaagaggttttgagccactttggaagaaccacaccggattatgatagcttgagccgcctcaagactataaccatgattctacatgaggtccttaggttgtacccaccggcaacctttctaacaagaagaacttataaggaaatggagctcggtggaatcaaatatcctgcaggagttgacctccttctacccgtcatcttcattcaccatgatcctgacatttggggaaaagacgcaagcgagttcaacccagaaaggtttgccaacggcatctccagcgcaaccaggcatcaggctgctttctttccgttcggaggggggcctagaatctgcatcggccagagctttgcgttgctggaagccaagatgacgctatgcaccatcctccaacgcttctcgttcgagctctcgccatcctacacccacgcgccgtacaccgtgataacactgcaccctcaacatggtgctcagataaggctcaaaaagctttctccgtgatgctccttctatgctgctaccggacactactttcgttactgaccgcatatgtagaaacgtatttcttatttagtatgtattttttaggatataaataaaaagatggtgcatattaatgggaaataagttcccttgtatgcattgcgatgtaattttgggaagatttggcaaggaacttaattatacaatatatgtattg

SEQ ID NO: 3 - DNA, 1539 bp, Arabidopsis thaliana var. Columbia
atgtctttttcagtagtagcagctttaccggtgcttgtagcagtagtggtactgtggacatggcggatcgtgaagtgggtctggataaaaccaaagatgcttgagagttctttgaaaagacagggtcttaccggaactccttacactcctctcgtcggagatataaagaggaatgttgatatgatgatggaagcgagatctaaacccatcaatgtaacggatgatatcaccccacgtctccttcctcttgccttaaagatgctcaattctcacggaaagactttcttcatatggattggaccacttccaacgattgtgataacgaatcctgagcagatcaaggaagtctttaataaagtcaacgactttgagaaagcttctacattccctttgatcagattgttagcaggtgggcttgcaagttacaagggagataaatgggcgagtcacaggaggatcatcaacccggcttttcacctcgaaaaaatcaagaacatgatccctgcgttctaccattgttgcagcgaggttgtctgtcaatgggagaagctatttacagataaagaatcgcctcttgaagtcgatgtttggccttggcttgtgaatatgactgcggatgtcatctcacatactgcttttggaagtagctataaagaagggcagagaatatttcaactacaaggggaattggctgagcttatcgcacaagcttttaagaaatcttacatccctggatcgaggttttacccaacaaagagcaatagaaggatgaaagcaatagatagagaagtagacgtaatattgagaggtattgtgagcaaacgggagaaagcgagagaagctggagaaccagcaaacgatgatttgttggggatattgcttgaatccaattcagaggaatctcaaggaaatggaatgagcgtagaagatgtgatgaaagagtgcaagttgttttatttcgcgggacaagagacaacttcagtacttttggtgtggactatggttttattaagccatcaccaagactggcaagctcgcgcacgagaggaagtgatgcaagtactcggtgaaaataataaacctgatatggagtcccttaacaaccttaaagtcatgactatgatcttcaatgaggttttgaggctataccctccggtggctcagcttaaacgagttgtcaacaaagaaatgaagctcggagagttgacccttccagctggaattcaaatttacttaccaactattcttgtccagcgtgacaccgagctttggggcgatgatgcagcggattttaaacccgagcggttcagagacgggctctcaaaggcaacaaagaaccaggtctctttcttcccctttggatggggacctaggatttgcatcggtcagaattttgctatgttggaggcaaagatggcaatggctttgattctacaaaagttctccttcgagctctctccttcttatgttcacgcgcctcaaacagtcatgaccactcgtccccaattcggagctcatctaattctgcacaagctctga

SEQ ID NO: 4 - DNA, 1539 bp, Arabidopsis thaliana var. Columbia
atggagatatcagtttcttcggtaacattttcactagctgtagttgttgtgtcttggtgggtatggagaacgttaaagtgggtttggttcacaccaaagatgcttgagcgttccctgagaagacaaggtctttccggaacttcttacacgcctctaatcggcgattttaaaaagatgatcagcatgttcattgaggcaacatccaaacccatcaaaccaacagatgatatcacccctcgtgtcatgcctcatcccttgcaaatgctcaagactcatggaaggactaacttaacatggtttggaccaataccaacaatcaccataatggatcctgagcaaatcaaggaagtgttcaacaaagtctatgacttccagaaggcgcatacgtttcctttaagcaaaatactaggcacgggactcgttagttatgatggcgataaatgggcgcaacaccgaagaatcatcaatccggctttccaccttgagaagatcaagaatatggtacatgtgttccacgaaagctgcagcgagcttgttggtgagtgggacaagttagtctcggataaagggtcctcatgtgaggtggacgtgtggcctgggcttacgagtatgactgcagatgtgatctcccgtactgcttttggtagcagctacagagaaggacacaggatatttgaactccaggcagaactagcacagctagtcatgcaagcttttcagaaattttttattcccggatatatttatctcccaacaaagggtaatagaaggatgaaaacagcagccagagaaatccaagatatactgagagggatcattaacaaaagggaaagggcaagagaatctggagaagcaccaagcgaggatttgctaggtatacttcttgaatcaaacttggggcaaacggaagggaatggaatgagtaccgaggatatgatggaagaatgcaagttgttctatttggccgggcaagagacaacatcagtacttctggtttggacaatggttctgttgagccaacaccaagattggcaggctcgtgcacgagaggaagtgaagcaagtttttggcgataaacaacctgatacagaaggccttaaccaactcaaagttatgacgatgatattatatgaggtccttaggctttatcctcctgtagtccagctgacccgagccattcacaaagagatgaagctcggagatctgactttaccaggcggtgttcagatcagtctacctgttctgcttgtccatcgcgacacggagctgtggggaaacgatgcaggggagttcaagcctgagagattcaaagacggcctctcaaaagcaacaaagaaccaagtctccttctttccctttgcgtggggaccaaggatctgcattggccagaattttacattgcttgaggcaaagatggcaatgagtttgattctacagagattctccttcgagctttctccttcctatgttcacgcgccttacacaatcatcaccctttacccacagttcggagctcatcttatgctgcacaagctctaa

SEQ ID NO: 5 - protein, 512 aa, Arabidopsis thaliana var. Columbia
MSFSVVAALPVLVAVVVLWTWRIVKWVWIKPKMLESSLKRQGLTGTPYTPLVGDIKRNVDMMMEARSKPINVTDDITPRLLPLALKMLNSHGKTFFIWIGPLPTIVITNPEQIKEVFNKVNDFEKASTFPLIRLLAGGLASYKGDKWASHRRIINPAFHLEKIKNMIPAFYHCCSEVVCQWEKLFTDKESPLEVDVWPWLVNMTADVISHTAFGSSYKEGQRIFQLQGELAELIAQAFKKSYIPGSRFYPTKSNRRMKAIDREVDVILRGIVSKREKAREAGEPANDDLLGILLESNSEESQGNGMSVEDVMKECKLFYFAGQETTSVLLVWTMVLLSHHQDWQARAREEVMQVLGENNKPDMESLNNLKVMTMIFNEVLRLYPPVAQLKRVVNKEMKLGELTLPAGIQIYLPTILVQRDTELWGDDAADFKPERFRDGLSKATKNQVSFFPFGWGPRICIGQNFAMLEAKMAMALILQKFSFELSPSYVHAPQTVMTTRPQFGAHLILHKL

SEQ ID NO: 6 - protein, 512 aa, Arabidopsis thaliana var. Columbia
MEISVSSVTFSLAVVVVSWWVWRTLKWVWFTPKMLERSLRRQGLSGTSYTPLIGDFKKMISMFIEATSKPIKPTDDITPRVMPHPLQMLKTHGRTNLTWFGPIPTITIMDPEQIKEVFNKVYDFQKAHTFPLSKILGTGLVSYDGDKWAQHRRIINPAFHLEKIKNMVHVFHESCSELVGEWDKLVSDKGSSCEVDVWPGLTSMTADVISRTAFGSSYREGHRIFELQAELAQLVMQAFQKFFIPGYIYLPTKGNRRMKTAAREIQDILRGIINKRERARESGEAPSEDLLGILLESNLGQTEGNGMSTEDMMEECKLFYLAGQETTSVLLVWTMVLLSQHQDWQARAREEVKQVFGDKQPDTEGLNQLKVMTMILYEVLRLYPPVVQLTRAIHKEMKLGDLTLPGGVQISLPVLLVHRDTELWGNDAGEFKPERFKDGLSKATKNQVSFFPFAWGPRICIGQNFTLLEAKMAMSLILQRFSFELSPSYVHAPYTIITLYPQFGAHLMLHKL

SEQ ID NO: 7 - DNA, 20 bp, Artificial Sequence
  JK6-29 PCR primer
cgttggaggatggtgcatag

SEQ ID NO: 8 - DNA, 21 bp, Artificial Sequence
  JK6-29 PCR primer
gctcggtggaatcaaatatcc

SEQ ID NO: 9 - DNA, 20 bp, Artificial Sequence
  Sequencing primer
gattcctgacccagagttag

SEQ ID NO: 10 - DNA, 20 bp, Artificial Sequence
  Sequencing primer
gcaaggagaactagctgaac

SEQ ID NO: 11 - DNA, 20 bp, Artificial sequence
  Sequencing primer
ccggattatgatagcttgag

SEQ ID NO: 12 - DNA, 17 bp, Artificial sequence
  Sequencing primer
cctgtgctttgcccatt

SEQ ID NO: 13 - DNA, 16 bp, Artificial Sequence
  Sequencing primer
cgcaaccaggcatcag

SEQ ID NO: 14 - DNA, 27 bp, Artificial Sequence
  BE10-30-3 PCR primer (BamH I)
atggatccatgctgcgggaagtctctc

SEQ ID NO: 15 - DNA, 28 bp, Artificial Sequence
  BE10-30-3 PCR primer (EcoR V)
acgatatcgtccggtagcagcatagaag

SEQ ID NO: 16 - DNA, 28 bp, Artificial Sequence
  CYP72A7 (Ath30) PCR primer (BamH I)
atcaggatccgtgagaatcggagatgtc

SEQ ID NO: 17 - DNA, 28 bp, Artificial Sequence
  CYP72A7 (Ath30) PCR primer (Kpn I)
tcgtggtaccaaatcagagcttgtgcag

SEQ ID NO: 18 - DNA, 29 bp, Artificial Sequence
  CYP72A14 (Ath29) PCR primer (Kpn I)
gtggtaccatggagatatcagtttcttcg

SEQ ID NO: 19 - DNA, 29 bp, Artificial Sequence
  CYP72A14 (Ath29) PCR primer (EcoR I)
ctgaattcatgtgattagagcttgtgcag

SEQ ID NO: 20 - DNA, 27 bp, Artificial Sequence
  BE10-30-3 PCR primer (Hpa I)
acgttaacatgctgcgggaagtctctc

SEQ ID NO: 21 - DNA, 28 bp, Artificial Sequence
  BE10-30-3 PCR primer (Xba I)
actctagaccggtagcagcatagaagga

SEQ ID NO: 22 - protein, 6 aa, Artificial Sequence
  Conserved motif
EVLRLY

SEQ ID NO: 23 - protein, 9 aa, Artificial Sequence
  Conserved motif
DVISXXAFG

SEQ ID NO: 24 - protein, 8 aa, Artificial Sequence
  Conserved motif
DXLGXXXX

(19) United States
(12) Patent Application Publication
(10) Pub. No.: US 2004/0103451 A1
(43) Pub. Date: May 27, 2004

(54) Abscisic acid 8'-and 7'-hydroxylase genes and related sequences from plants

(76) Inventors: Joan E. Krochko, Saskatoon (CA); Adrian J. Cutler, Saskatoon (CA); Suzanne R. Abrams, Saskatoon (CA)

Correspondence Address: KIRBY EADES GALE BAKER, BOX 3432, STATION D, OTTAWA, ON, K1P 6N9, CA

(21) Appl. No.: 10/432,609

(22) PCT Filed: Dec. 6, 2001
(86) PCT No.: PCT/CA01/01756

(51) Int. Cl. A01H 1/00; C12N 15/82; C07H 21/04; C12N 9/02

(52) U.S. Cl. 800/278; 435/189; 435/468; 536/23.2

(57) ABSTRACT

The present invention relates to plant enzymes responsible for hydroxylation of abscisic acid, said enzymes capable of 7′- and 8′-hydroxylation of abscisic acid, and polynucleotides encoding the same. The invention further relates to methods for using said polynucleotides to alter plant metabolism, in particular metabolism of abscisic acid. The invention further relates to plants exhibiting altered characteristics as a result of the alteration of abscisic acid metabolism.

### BACKGROUND OF THE INVENTION

1. Field of the Invention

The present invention relates to plant enzymes responsible for the inactivation of the biological activity of abscisic acid, said enzymes capable of 7′- and 8′-hydroxylation of abscisic acid, polynucleotides encoding the same and methods to use said polynucleotides to alter plant metabolism.

2. Background Art

The phytohormone S-(+)-abscisic acid (ABA) plays regulatory roles in a host of physiological processes in all higher as well as lower plants. ABA mediates stress tolerance responses in higher plants, is a key signal compound that regulates water loss or transpiration and, in concert with other plant signaling compounds, is implicated in mediating responses to pathogens and wounding. In plant seeds, ABA promotes seed development, desiccation tolerance, and inhibition of germination. ABA affects plant architecture, including root growth and morphology, root-to-shoot ratios, and plant fertility and reproduction.

The plant growth regulator ABA has been the subject of study for many years. However little is known in regarding the control of ABA levels or the specific mechanisms by which ABA mediates its activity.

Specific physiological processes that are known to be mediated at least in part by ABA include those related to a number of important agronomic characteristics.

ABA concentrations are important for stages of embryogenesis, and for plant growth and development in general. Plant architecture, size and yield are affected by levels and metabolism of ABA. Examples of the potential effects of manipulating ABA levels follow.

Increased ABA levels have been shown to increase expression of genes involved in lipid accumulation in microspore-derived B. napus embryos.

Experiments utilizing long lasting ABA analogs (i.e. ABA analogs that act as ABA agonists and are not subject to degradation by 8′-hydroxylation, Abrams S. R., et al., 1997, Plant Physiol 114: 89-97) provide a prolonged, higher intensity ABA-like signal than that produced by ABA itself, with resulting increases in oil accumulation relative to untreated and ABA-treated embryos (Qi Q., et al., 1998, Plant Physiol 117: 979-987). Thus ABA activity has been clearly demonstrated to have important and potentially beneficial effects on oil accumulation in oilseeds. Therefore increasing ABA content of embryos by reducing catabolism is likely to be beneficial in terms of increased oil accumulation.

In addition to oil accumulation, seed protein accumulation, including storage proteins, is dependent upon ABA in many crop species. For example, in B. napus the accumulation of seed proteins (napin, oleosin and cruciferin) including late embryogenesis abundant proteins (LEA's), increases in response to ABA treatments. In maize and wheat, globulin accumulation is dependent on ABA activity (Bewley J. D. and Black M., 1994, Seeds: Physiology of Development and Germination. Plenum Press, New York and London). Therefore ABA plays an important role in storage protein biosynthesis in seeds.

ABA promotes maturation and desiccation tolerance in seeds, and high ABA levels accelerate seed maturation (Green B. R., et al., 1998, Physiol Plantarum 104: 125-133).

ABA promotes maturation and desiccation tolerance in seeds, so seed maturation will be accelerated by alteration of ABA activity (Johnson-Flanagan A. M., et al., 1992, Plant Physiol 99: 700-706). An aspect of this function in canola is that ABA is implicated in chlorophyll clearing in maturing B. napus seed (Green et al., ibid). The green seed problem in oilseed canola (persistence of seed chlorophyll after frosts leading to discolored oil) can be potentially modulated by controlling ABA levels.

In response to abiotic stresses such as cold, heat and drought, levels of ABA rise in plants, triggering biochemical and physiological responses to accommodate the stress. ABA directly causes closure of stomata and reduced transpiration. Mutants of Arabidopsis thaliana have been produced that have increased sensitivity to ABA (Bonetta D. and McCourt P., 1998, Trends Plant Science 3: 231-235) and appear more stress tolerant. Combining this hypersensitivity to ABA with increased ABA levels during perception of stress may lead to a further enhancement of stress tolerance in transgenic crops such as canola.

Genetic and physiological data indicates that ABA is involved in maintaining and inducing dormancy as well inhibiting germination (Bewley and Black, ibid, Bonetta and McCourt, ibid). Therefore germination of seeds can be inhibited by ABA, whether supplied exogenously or synthesized in the seed.

Inappropriate dormancy regulation causes problems in a variety of crops in specific situations. These problems may be abolished by fine-tuning ABA levels in seeds. Preharvest sprouting in cereals is a problem that occurs when germination of the seed in the head proceeds prematurely and is facilitated by high moisture levels. The problem is acute for malting barley that is bred for low dormancy.

An example of a situation in which fine-tuning the seed dormancy of a crop would be advantageous is provided by fall seeding of annual crops. Fall seeding of some normally spring-sown annual crops (e.g. spring canola), affords early spring germination and emergence resulting in a longer season for plant maturity. Early emergence also allows the seedling to take better advantage of accumulated winter moisture and avoid pests and pathogens. Flowering can occur earlier in the season and heat stress is avoided. However, premature fall germination (of spring crops sown in the fall) can result in counterproductive winterkill. Therefore adjusting ABA levels so that seed remains dormant in the fall, but germinates relatively early in the following spring would allow growers to take full advantage of fall seeding.

Exogenous application of ABA and related ABA analogs have been shown to cause a shift in the ratio of shoot to root growth, favouring increased root growth. Therefore, increased endogenous ABA levels could result in altering the balance in crop plants to permit greater root growth and better nutrient and water uptake.

“Crosstalk” or interactive, cooperative and combined effects between plant growth regulators and other signaling molecules including sugars have been documented (Moller S. G. and Chua N-H., 1999, J Mol Biol 293: 219-234). Auxins are thought to work through ethylene and ABA. Thus ABA plays an important role in the physiological control of plant developmental processes and influences processes not normally specifically associated with ABA action. For example, ABA influences the ability to set flowers, therefore alteration in ABA metabolism has consequences for male and female sterility and seed production.

The biosynthesis and metabolism of ABA is only partly understood (Cutler A. J. and Krochko J. E., 1999, Trends in Plant Science 4: 472478). ABA is produced within plant cells in response to physiological and developmental signals. As evidenced above, once produced, ABA affects a number of physiological processes important to plant growth and development. ABA is one of the primary plant growth regulators that have been the object of study for some time. ABA, in conjunction with the plant growth regulators auxins, cytokinins, brassinosteroids, gibberellins and ethylene represent the predominant molecules for the hormonal control of plant growth. Hence the control of ABA synthesis and degradation is an important factor in the control of plant growth and development.

In order to improve plant performance and increase plant productivity, it is advantageous to manipulate plant growth regulator concentrations as a means to control plant growth. Typically manipulation of the effects of plant growth regulators has been through the development and application of analogs, development and use of compounds that inhibit the production or transport of plant growth regulators, or the application of synthetic versions of plant growth regulators.

Alternatively it is possible to manipulate the response of plant cells by inhibition or alteration of the natural metabolism of plant hormones, thus altering the natural levels by either increasing or decreasing the rate at which the growth regulator is metabolized. For naturally occurring plant growth regulators, plant cells typically modulate the levels of these substances by balancing synthesis and degradation (Cutler A. J. and Krochko J. E., 1999, TIPS 4: 472-478, Zeevaart J. A. D., 1999, In Biochemistry and Molecular Biology of Plant Hormones, pp189-207).

A primary control point for maintaining growth regulator levels in plant cells is believed to be the metabolism through control of enzymatic activity responsible for the biochemical conversion of a plant growth regulator to an inactive or substantially less active compound. The degradation of plant growth regulators provides a mechanism for the plant cell to rapidly alter levels of specific regulators in response to physiological conditions. This mechanism, which is usually mediated by a specific enzyme activity, can be regulated at a number of different levels including gene expression of the catabolic enzyme, specific manipulation of enzyme activity through allosteric regulation or availability of co-factors, as well as the turnover of the enzyme itself. The degradation of ABA is one of the primary control points for the mediation of ABA activity in plant cells.

ABA, once produced or transported into a plant cell is metabolized very quickly. The molecule is susceptible to rapid enzymatic degradation through oxidative catabolism to 8′-hydroxyABA, with subsequent conversion to (−)-Phaseic Acid (PA) and in some tissues reduction to (−)-Dihydrophaseic Acid (DPA). These latter compounds are generally not as active as ABA in mediating physiological responses, thus, as a consequence of these conversions the effectiveness of ABA synthesized in, or imported into tissues is reduced. These compounds derived by catabolism of ABA can then be further catabolized within the cell.

The key step in the catabolism of ABA is the hydroxylation activity. It is believed that an 8′-hydroxylase activity is the predominant means for inactivation and catabolism of ABA, but it has also been postulated that a 7′-hydroxylase can produce (+)-7′-hydroxyABA from (+)-ABA. This process also leads to inactive compounds and hence conversion of ABA to 7′-hydroxyABA is another mechanism for the catabolism of ABA. It has also been postulated that a reductase activity may function in the conversion of ABA to ABA cis-1′,4′-diol, the extent to which this operates in vivo is unknown. In addition, the formation of inactive ABA conjugates through the activity of a glucosyltransferase also has been implicated as a means of modulating concentrations of hormonally active ABA in plant cells. Nonetheless, the published evidence suggests that the primary pathway for degradation of ABA is through hydroxylation, and the predominant activity appears to be the 8′-hydroxylase activity (Cutler and Krochko, ibid).

Experimental evidence implicates the turnover of ABA to be a critical factor in controlling ABA responses in plants and mediation of ABA effects. Some of the metabolic markers used to identify tissues expressing ABA hydroxylase enzymes include the disappearance of internal or externally applied ABA, accumulation of PA and DPA, and reduced sensitivity to applied ABA (Uknes S. J. and Ho T. H. D., 1984, Plant Physiol 75: 1126-1132, Garello G. and LePage-Degivry M. T., 1995, Plant Physiol 95: 45-50, Jia W., et al., 1996, J Exp Bot 47: 1085-1091, Cutler A. J., et al., 1997, J Exp Bot 48: 1787-1795, Qi Q., et al., 1998, Plant Physiol 117: 979-987).

ABA degradation can be very rapid, the half-life of applied ABA has been observed to be 42 and 60 minutes in leaves of maize and Commelina communis, respectively (Jia et al., ibid), and in isolated maize root tips the turnover was greater than 60% in one hour (Ribaut J. M., et al., 1996, J Plant Physiol 148: 761-764).

Evidence suggests that ABA 8′-hydroxylase activity is induced by ABA and possibly regulated by osmotic stress and phytochrome-dependent signaling pathways (Cutler et al., ibid, Kraepiel Y., et al., 1994, Plant J 6: 665-672). Enzyme characterization indicates that ABA 8′-hydroxylase is a member of the cytochrome P450 monooxygenase superfamily (Krochko J. E., et al., 1998, Plant Physiol 118: 849-860).

The purification of ABA 8′-hydroxylase has not been previously achieved, thus information on the protein structure and genetic characterization of the enzyme is lacking. However, general studies on characterization of ABA 8′-hydroxylase activity in tissue extracts have provided evidence that the enzyme is a cytochrome P450 monooxygenase (see Krochko et al., 1998, Plant Physiol 118: 849-860). In these studies ABA 8′-hydroxylase activity 10 (determined by in vitro assay) is transient in Black Mexican Sweet corn suspension cells (BMS cells) with a peak of activity at 16 hours after the addition of (+)-ABA to the BMS corn suspension culture medium. It was found that ABA 8′-hydroxylase is a microsomal enzyme; ABA 8′-hydroxylase requires NADPH; ABA 8′-hydroxylase requires O₂; ABA 8′-hydroxylase is inhibited by CO, tetcyclacis and oxidized cytochrome c; the CO inhibition of ABA 8′-hydroxylase is relieved by blue-light, all of these criteria showing that ABA 8′-hydroxylase is a cytochrome P450 monooxygenase.

Other studies (see Cutler et al., 1997, J Exp Bot 48: 277-287) have concluded that: the ABA 8′-hydroxylase is induced by (+)-ABA (its substrate) in maize BMS suspension cultured cells; ABA 8′-hydroxylase expression in corn suspension cells is short-lived even in the continuing presence of the inducing compound, (+)-ABA; induction of ABA 8′-hydroxylase in corn suspension cells is inhibited by cycloheximide (a protein synthesis inhibitor) and cordecypin (an RNA synthesis inhibitor). These data suggest that this enzyme activity is transcriptionally regulated, i.e. (+)-ABA induces the synthesis of new mRNA for ABA 8′-hydroxylase.

Thus, biochemical characterization of enzyme requirements, appearance and overall enzyme characteristics indicate that the 8′-hydroxylase activity responsible for the hydroxylation of ABA is a member of the P450 monooxygenase family, the expression of which is induced by the occurrence of ABA itself. The polypeptide sequence of the enzyme itself has remained elusive since many P450 monooxygenase enzymes have proved difficult to purify. Thus a gene encoding an ABA 8′-hydroxylase activity, a 7′-hydroxylase activity, or any ABA hydroxylase activity has not been described in the art.

However, as more DNA sequence, and inferred protein sequences have become available from gene sequencing studies, many P450 monooxygenase coding sequences have been identified. There are a significant number of P450 genes that have been identified by sequencing, however, the function of the majority of these sequences remains unknown. Most of the sequences have not yet been ascribed a function in terms of plant metabolism or more relevant to the present problem, their role in ABA hydroxylation. Thus, the art does not provide a means to identify an ABA hydroxylase activity within the P450 monooxygenase family.

Plant cytochrome P450 monooxygenases (excluding CYPS 1) are named from CYP71—CYP99 and from CYP701 upwards as new discoveries are made. There are currently 273 P450 genes named according to this scheme in 20 Arabidopsis thaliana (http://drnelson.utmem.edu/Arabfam.html) comprising 45 distinct P450 families. Functions have been ascribed to less than 30 of these P450 genes. At present, the genes encoding an ABA 8′ or 7′-hydroxylase gene has not been identified.

The search for the specific P450 gene sequence encoding an ABA hydroxylase has been undertaken in many laboratories. Many genetic screens of mutant plant lines have been carried out that should have led to the isolation of an ABA 8′-hydroxylase gene. However, these studies have not been successful and this suggests that mutants in the ABA 8′-hydroxylase gene could be lethal, or that this enzyme activity is coded by a multigene family. Thus the gene encoding the enzyme has not been unequivocally identified.

As a result of the novel screening methodology employed, and on the basis of the genetic sequences obtained by this method, the present inventors have identified a novel ABA hydroxylase gene. The results presented in this specification suggest that genes responsible for ABA hydroxylase activities are part of a multigene family with an inherent redundancy of gene function, which has prevented this gene from being isolated by current genetic methods. The methods employed within the scope of this invention have allowed these limitations to be overcome and hence led to the isolation of this genetic activity encoding an ABA hydroxylase.

### 4. SUMMARY OF THE INVENTION

It is the object of the present invention to provide a P450 monooxygenase enzyme encoding an ABA hydroxylase activity.

It is another object of the present invention to provide a polynucleotide sequences encoding an ABA 8′-hydroxylase activity.

It is another object of the present invention to provide a polynucleotide sequences encoding an ABA 7′-hydroxylase activity.

It is the object of the present invention to provide a polynucleotide sequence encoding an ABA hydroxylase activity from corn cells.

It is still another object of the present invention to provide polynucleotide sequences homologous to ABA 7′-hydroxylase and ABA 8′-hydroxylase sequences from a variety of plant species. These sequences are identified by homology to the sequences of the CYP72A P450 monooxygenase family that have been now identified as encoding ABA hydroxylase activity.

It is another object of the present invention to provide a means to utilize ABA hydroxylase activities to alter plant metabolism through the modified catabolism of endogenously produced ABA.

It is another object of the present invention to provide a means to utilize ABA 7′-hydroxylase and ABA 8′-hydroxylase sequences to modify plant metabolism by alteration of the activity of exogenously applied ABA.

It is another object of the present invention to provide a means to utilize ABA 7′-hydroxylase and ABA 8′-hydroxylase sequences to modify plant metabolism by alteration of the activity of exogenously applied ABA analogs.

The present invention provides nucleic acid sequences encoding a novel hydroxylase enzyme involved in catabolism of ABA. The pBE10-30-3 cDNA sequence encodes an ABA hydroxylase gene that is expressed following ABA induction in plant cells. The gene sequence is useful for identification of related sequences from other plant species.

According to one aspect of the invention, there is provided an isolated polynucleotide comprising a nucleotide sequence encoding a polypeptide of a plant P450 monooxygenase family capable of hydroxylating ABA. The polynucleotide may be DNA or RNA.

According to another aspect of the invention, there is provided an isolated polynucleotide comprising SEQ ID NO:1 or the complement of SEQ ID NO:1 or a sequence homologous to SEQ ID NO:1 or the complement of SEQ ID NO:1.

According to another aspect of the invention, there is provided an isolated polynucleotide comprising SEQ ID NO:2 or the complement of SEQ ID NO:2, or a sequence homologous to SEQ ID NO:2 or the complement of SEQ ID NO:2.

According to another aspect of the invention, there is provided an isolated polynucleotide comprising SEQ ID NO:3 or the complement of SEQ ID NO:3, or a sequence homologous to SEQ ID NO:3 or the complement of SEQ ID NO:3.

According to another aspect of the invention, there is provided an isolated polynucleotide comprising SEQ ID NO:4 or the complement of SEQ ID NO:4, or a sequence homologous to SEQ ID NO:4 or the complement of SEQ ID NO:4.

In another aspect, the invention provides an isolated polynucleotide comprising a sequence encoding a ABA 8′-hydroxylase enzyme of a plant or a protein having the same enzymatic activity thereas.

In another aspect, the invention provides an isolated polynucleotide comprising a sequence encoding an ABA 7′-hydroxylase enzyme of a plant or a protein having the same enzymatic activity thereas.

In yet another aspect, the invention provides a chimeric gene comprising a polynucleotide sequence of the type described above, operably linked to suitable regulatory sequences.

In yet another aspect, the invention provides a method for modifying the ABA catabolism of a plant comprising: (a) introducing into a plant cell capable of being transformed and regenerated to a whole plant a genetic construct comprising a first DNA expression cassette that comprises, in addition to the DNA sequences required for transformation and selection in plant cells, a DNA sequence that encodes a polynucleotide as described above, operably linked to a suitable transcriptional regulatory region, and (b) recovery of a plant which contains said recombinant DNA. The polynucleotide may be in the sense or the antisense orientation relative to the transcriptional regulatory region.

In yet another aspect, the invention provides a method for modifying the ABA catabolism of a plant comprising: (a) introducing into a plant cell capable of being transformed and regenerated to a whole plant a genetic construct comprising a first DNA expression cassette that comprises, in addition to the DNA sequences required for transformation and selection in plant cells, a DNA sequence that encodes a polynucleotide as described above, operably linked to a transcriptional regulatory region that can be induced for expression and (b) recovery of a plant which contains said recombinant DNA. The polynucleotide may be in the sense or the antisense orientation relative to the transcriptional regulatory region.

In yet another aspect, the invention provides a method for modifying the catabolism of a plant comprising: (a) introducing into a plant cell capable of being transformed and regenerated to a whole plant a genetic construct comprising a first DNA expression cassette that comprises, in addition to the DNA sequences required for transformation and selection in plant cells, a DNA sequence that encodes a polynucleotide as described above, operably linked to a transcriptional regulatory region that is tissue specific, and (b) recovery of a plant which contains said recombinant DNA. The polynucleotide may be in the sense or the antisense orientation relative to the transcriptional regulatory region.

Another object of the present invention relates to the use of a polynucleotide comprising a nucleotide sequence encoding a polypeptide of a plant P450 monooxygenase family capable of hydroxylating ABA analogs.

In this aspect of the invention, there is provided a method for modifying the catabolism of ABA analogs in a plant cell comprising: (a) introducing into a plant cell capable of being transformed and regenerated to a whole plant a genetic construct comprising a first DNA expression cassette that comprises, in addition to the DNA sequences required for transformation and selection in plant cells, a DNA sequence that encodes a polynucleotide as described above, operably linked to a suitable transcriptional regulatory region, (b) recovery of a plant which contains said recombinant DNA, and exposing said plant cell to an ABA analog.

Said transcriptional control region may be constitutive, conditional or tissue specific in its expression pattern.

In a still further embodiment of the invention, the plant cell described above is induced to regenerate a whole plant and the whole plant comprising a novel ABA hydroxylase activity is treated with an ABA analog plant exhibiting different physiological responses at the whole plant level when compared to a plant without the novel ABA hydroxylase gene being exposed to said ABA analog.

The invention also relates to plants and plant parts transformed with a chimeric gene as described above.

The pJK6-29 (SEQ ID NO:1) and pBE10-30-3 (SEQ ID NO:2) sequence and related sequences CYP72A7 (SEQ ID NO:3) and CYP72A14 (SEQ ID NO:4) are used to modify catabolism of ABA by the transformation of plant cells with a plant transformation vector comprising a sense or antisense portion of the gene or a double stranded RNA comprising of both sense and antisense portions of the gene.

The pJK6-29 and pBE10-30-3 sequence may also be used for identification of related homologous sequences deposited in public databases through comparative techniques well-known in the art, or as hybridization probes for the identification of related cDNA or genomic sequences from various plant species where the DNA sequence information is not known.

The invention also relates to protein sequences encoding ABA hydroxylase activity, said protein sequences having at least 50% protein identity to the protein encoded by SEQ ID NO:2.

The invention also relates to proteins encoding an ABA hydroxylase activity, said proteins having conserved amino acid regions identified as ‘EVLRLY’ (SEQ IS NO.22) and ‘DVIS[KRH]xAFG’ (SEQ ID NO.23) as described herein.

### 5. BRIEF DESCRIPTIONS OF THE DRAWINGS

FIG. 1. Evaluation of the subtractive cloning procedure for the ability to select cDNA uniquely expressed in response to ABA induction (i.e. assessment of the quality of the PCR suppression subtraction (Clonetech)). Comparison of the cDNA populations following the subtractive procedure. Four cDNA samples (NI/S; non-ABA-induced and subtracted, NI/N; non-ABA-induced and non-subtracted, IND/S; ABA-induced and subtracted, IND/N; ABA-induced and non-subtracted) resulting from the subtractive hybridization were amplified by PCR and equivalent amounts were loaded onto an agarose gel. After transfer to nylon membrane the blot was probed successively with radiolabeled rice actin cDNA and subtracted ABA-induced cDNA (IND/S). Probes: a) Rice Actin; b) Subtracted ABA-Induced cDNA (IND/S); c) Ethidium Bromide.

FIG. 2. Isolation of an ABA-induced cytochrome P450 monooxygenase cDNA by screening a population of subtracted cDNA with probes from subtracted induced and subtracted non-induced tissues. The figure illustrates randomly selected cDNA clones from a subtracted ABA-induced library (PCR-amplified inserts of 48 clones arrayed in dulpicate/blot). The PCR-amplified inserts were arrayed in duplicate on nylon membranes and probed with labeled subtracted non-ABA-induced cDNA (NI/S) and subtracted ABA-induced cDNA (IND/S). Clones that showed at least a 3 to 5-fold greater intensity of labeling with the IND/S cDNA probe were deemed to be upregulated by ABA and DNA sequences were determined. One of these, pJK6-29, indicated with a box on the nylon cDNA array represents a C-terminal fragment of a cytochrome P450 monooxygenase. a) Subtracted non-induced cDNA probe (NI/S); b) Subtracted induced cDNA probe (IND/S).

FIG. 3. Southern blot analysis of cDNA populations obtained from induced and non-induced cells using the P450 cDNA clone pJK6-29 (333 bp, C-terminal). The isolated P450 cDNA (pJK6-29) was used as a probe against the four cDNA pools resulting from the subtractive hybridization procedure. This cytochrome P450 was upregulated by ABA (compare NI/N and IND IN) and successfully amplified within the subtracted ABA-induced cDNA pool (compare NI/S and IND/S).

FIG. 4. Northern blot analysis with P450 cDNA pJK6-29 (333 bp, C-terrminal). Total RNA isolated from non-ABA-induced and ABA-induced (9 hr at 200 μM (+)-ABA) BMS corn suspension was probed with radiolabeled pJK6-29 to examine the regulation of expression by (+)-ABA in corn suspension cells.

FIG. 5. Comparison of ABA 8′-hydroxylase activity and expression of pJK629 mRNA after induction of plant cells with (+)-ABA. ABA 8′-hydroxylase activity was determined in BMS corn suspension cells at defined periods after the addition of 200 μM (+)-ABA. Total RNA was extracted from duplicate tissue samples and expression of the P450 clone, pJK6-29, was determined by Northern blot analysis. a) ABA 8′-hydroxylase activity; b) mRNA accumulation JK6-29 (P450).

FIG. 6. Heterologous expression of an Arabidopsis P450 ABA 7′-hydroxylase activity in yeast. The CYP72A7 cDNA from Arabidopsis thaliana was inserted into the pYeDP60 vector, transformed into the WAT11 yeast strain (but not the WAT21 yeast strain) and analyzed for the ability to metabolize (+)-ABA. The biotransformation of (+)-ABA to 7′-hydroxyABA was assessed by High Performance Liquid Chromatography—Mass Spectrometry. (HPLC/MS/MS) 7′-hydroxyABA was excreted into the media. Measured activity was limited by access of enzyme to the substrate (ABA).

FIG. 7. Identification of the EVLRLY (SEQ ID NO.22) conserved amino acid sequence in CYP72A genes in plants. This figure illustrates the position of the conserved amino acid sequence found in all CYP72A genes, including those identified as encoding ABA hydroxylase activity. Conserved regions are boxed. Position of the conserved sequence is shown relative to the amino acid residue number shown on the right hand side of the listing. The specific genes encoding the protein sequences are shown on the left hand side.

FIG. 8. Identification of the ‘DVIS[KRH]xAFG’ (SEQ ID NO.23) conserved amino acid sequence in CYP72A genes in plants. This figure illustrates the position of the conserved amino acid sequence found in all CYP72A genes, including those identified as encoding ABA hydroxylase activity. Conserved regions are boxed. Position of the conserved sequence is shown relative to the amino acid residue number shown on the right hand side of the listing. The specific genes encoding the protein sequences are shown on the left hand side.

FIG. 9. Construction of the Yeast pYeDP60.CornP450 expression plasmid

FIG. 10. Construction of the Yeast pYeDP60.Ath30-2 expression plasmid.

FIG. 11. Construction of the Yeast pYeDP60.Ath29-3 expression plasmid FIG. 12. Construction of the binary vectors, pCAMBIA2300.Ath30-2Forward and pCAMBIA2300.Ath30-2Reverse. In a first step, the 35S.Ath30-2Forward cassette or the 35S.Ath30-2Reverse cassette was cut out with Xho I, blunt ended and then cut with Kpn I.

FIG. 13. Construction of the binary vectors, pCAMBIA2300.Ath29-3Forward and pCAMBIA2300.Ath29-3Reverse. In a first step, the 35S.Ath29-3Forward cassette or the 35S.Ath29-3Reverse cassette was cut out with Xho I, blunt ended and then cut with Kpn I.

FIG. 14. Construction of the pJIT61.CornP450Forward plasmid.

FIG. 15. Construction of the p35S.CAMBIA2301 plasmid.

FIG. 16. Construction of the pCAMBIA2301.CornP450Forward plasmid. Initially, p35S.CAMBIA2301 was cut with Pst I, blunted ended and cut with Bam HI. In addition, pJIT61.CornP450Forward was cut with BamHI and EcoR V.

FIG. 17. Construction of the pJIT61.CornP450Reverse plasmid.

FIG. 18. Construction of the binary vector, CAMBIA2301.CornP450Reverse. Initially, p35S.CAMBIA2301 was cut with Pst I, blunt-ended and cut with Xba I. In addition, pJIT61.CornP450Reverse was cut with Xba I and EcoR V.

FIG. 19. Construction of the plasmid for tetracycline-inducible expression, pBINHygTx.Ath30-2. Initially, pYeD60.Ath30-2 was cut with BamH I, blunt-ended and cut with Kpn I.

FIG. 20. Construction of the plasmid for tetracycline-inducible expression, pBINHygTx.Ath29-4.

FIG. 21. Construction of the plasmid for tetracycline-inducible expression, pBINHygTx.CornP450.

### 6. DETAILED DESCRIPTION OF THE INVENTION

Definitions

In order to provide a clear and consistent understanding of the specification and claims, including the scope to be given certain terms used therein, the following definitions are provided.

The singular forms “a,” “an,” and “the” include plural reference unless the context clearly dictates otherwise.

An analog of abscisic acid is defined as a molecule structurally resembling abscisic acid comprised of a six-membered ring with a side chain containing two units of unsaturation. The ring can have a double bond like abscisic acid but also may be saturated. The methyl groups of the ring may be replaced with hydrogen, chains or rings containing unsaturated carbon-carbon functional groups, or heteroatoms. The oxygens of ABA can be altered in oxidation state or can be replaced with hydrogen or other functional groups. Analogs can confer activity similar to abscisic acid or can antagonize ABA effects.

A “coding sequence” or “coding region” is the part of a gene that codes for the amino acid sequence of a protein, or for a functional RNA such as a tRNA or rRNA. A coding sequence typically represents the final amino acid sequence of a protein or the final sequence of a structural nucleic acid. Coding sequences may be interrupted in the gene by intervening sequences, typically intervening sequences are not found in the mature coding sequence.

A “complement” or “complementary sequence” is a sequence of nucleotides which forms a hydrogen-bonded duplex with another sequence of nucleotides according to Watson-Crick base-pairing rules. For example, the complementary base sequence for 5′-AGCT-3′ is 3′-TCGA-5′.

“Expression” refers to the transcription of a gene into structural RNA (rRNA, tRNA) or messenger RNA (mRNA) with subsequent translation into a protein in the case of the mRNA.

Polynucleotides are “functionally equivalent” if they perform substantially the same biological function. By substantially the same biological function it is meant that similar protein activities or protein function are encoded by a mRNA polynucleotide, or a structural polynucleotide has a similar structure and biological activity

Polynucleotides are “heterologous” to one another if they do not naturally occur together in the same arrangement in the same organism. A polynucleotide is heterologous to an organism if it does not naturally occur in its particular form and arrangement in that organism.

Polynucleotides or polypeptides have “homologous” or “identical” sequences if the sequence of nucleotides or amino acid residues, respectively, in the two sequences is the same when aligned for maximum correspondence as described herein. Sequence comparisons between two or more polynucleotides or polypeptides are generally performed by comparing portions of the two sequences over a portion of the sequence to identify and compare local regions. The comparison portion is generally from about 20 to about 200 contiguous nucleotides or contiguous amino acid residues or more. The “percentage of sequence identity” or “percentage of sequence homology” for polynucleotides and polypeptides, such as 50, 60, 70, 80, 90, 95, 98, 99 or 100 percent sequence identity may be determined by comparing two optimally aligned sequences which may or may not include gaps for optimal alignment over a comparison region, wherein the portion of the polynucleotide or polypeptide sequence in the comparison may include additions or deletions (i.e., gaps) as compared to the reference sequence (which does not comprise additions or deletions) for optimal alignment of the two sequences.

The percentage of homology or similarity is calculated by: (a) determining the number of positions at which the identical nucleic acid base or amino acid residue occurs in both sequences to yield the number of matched positions; (b) dividing the number of matched positions by the total number of positions in the window of comparison; and, (c) multiplying the result by 100 to yield the percentage of sequence identity.

Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul S. F., et al., 1990, J Mol Biol 215: 403, Altschul S. F., et al., 1997, Nucleic Acids Res 25: 3389-3402) and ClustalW programs. BLAST is available on the Internet at http://www.ncbi.nlm.nih.gov and a version of ClustalW is available at http://www2.ebi.ac.uk. Other suitable programs include GAP, BESTFIT, FASTA, and TFASTA in the Wisconsin Genetics Software Package (Genetics Computer Group (GCG), 575 Science Dr., Madison, Wis.). For greater certainty, as used herein and in the claims, “percentage of sequence identity” or “percentage of sequence homology” of amino acid sequences is determined based on optimal sequence alignments determined in accordance with the default values of the BLASTX program, available as described above.

Sequence identity typically refers to sequences that have identical residues in order, whereas sequence similarity refers to sequences that have similar or functionally related residues in order. For example an identical polynucleotide sequence would have the same nucleotide bases in a specific nucleotide sequence has found in a different polynucleotide sequence. Sequence similarity would include sequences that are similar in character for example purines and pyrimidines arranged in a specific fashion. In the case of amino acid sequences, sequence of identity means the same amino acid residues in a specific order, where as sequence similarity would allow for amino acids with similar chemical characteristics (for instance basic amino acids, or hydrophobic amino acids) to reside within a specific order. The terms “stringent conditions” or “stringent hybridization conditions” includes reference to conditions under which a probe will hybridize to its target sequence, to a detectably greater degree than other sequences (e.g. at least 2-fold over background). Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength and pH) at which 50% of a complementary target sequence hybridizes to a perfectly matched probe. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. Exemplary low stringency conditions include hybridization with a buffer solution of 30% formamide, 1 M NaCl, 1% SDS at 37° C., and a wash in 2×SSC at 50° C. Exemplary high stringency conditions include hybridization in 50% formamide, 1 M NaCl, 1% SDS at 37° C., and a wash in 0.1×SSC at 60° C. Hybridization procedures are well-known in the art and are described in Ausubel et al., (Ausubel F. M., et al., 1994, Current Protocols in Molecular Biology, John Wiley & Sons Inc.).

“Isolated” refers to material that is: (1) substantially or essentially free from components which normally accompany or interact with it as found in its naturally occurring environment; or (2) if in its natural environment, the material has been non-naturally altered to a composition and/or placed at a locus in the cell not native to a material found in that environment. The isolated material optionally comprises material not found with the material in its natural environment. For example, a naturally occurring nucleic acid becomes an isolated nucleic acid if it is altered, or if it is transcribed from DNA which is altered, by non-natural, synthetic methods performed within the cell from which it originates.

Two DNA sequences are “operably linked” if the linkage allows the two sequences to carry out their normal functions relative to each other. For instance, a promoter region would be operably linked to a coding sequence if the promoter were capable of effecting transcription of that coding sequence.

A “polynucleotide” is a sequence of two or more deoxyribonucleotides (in DNA) or ribonucleotides (in RNA).

A “DNA construct” is a nucleic acid molecule that is isolated from a naturally occurring gene or which has been modified to contain segments of nucleic acid which are combined and juxtaposed in a manner which would not otherwise exist in nature.

A “polypeptide” is a sequence of two or more amino acids.

A “promoter” or transcriptional regulatory region is a cis-acting DNA sequence, generally located upstream of the initiation site of a gene, to which RNA polymerase may bind and initiate correct transcription.

A “recombinant” polynucleotide, for instance a recombinant DNA molecule, is a novel nucleic acid sequence formed through the ligation of two or more nonhomologous DNA molecules (for example a recombinant plasmid containing one or more inserts of foreign DNA cloned into it).

“Transformation” means the directed modification of the genome of a cell by the external application of recombinant DNA from another cell of different genotype, leading to its uptake and integration into the subject cell's genome.

A “transgenic plant” encompasses all descendants, hybrids, and crosses thereof, whether reproduced sexually or asexually, and which continue to harbour the foreign DNA.

Unless defined otherwise all technical and scientific terms used herein have the same meaning as commonly understood to one of ordinary skill in the art to which this invention belongs.

Isolation of ABA Hydroxylase Sequences

The present invention describes a nucleic acid sequences that encode enzyme activities capable of hydroxylating ABA. These nucleic acid sequences are members of the P450 monooxygenase family. Specifically these nucleic acid sequences are members of the CYP72A gene subfamily.

These genes were isolated based on a number of characteristics that are indicative of an ABA hydroxylase activity. It is known that there are a number of P450 monooxygenase genes that have been described in plants. Those P450 monooxygenase genes that encode ABA hydroxylase activity have been found to be limited to a subset of the P450 gene family as described herein. Biochemical evidence of the function of these genes has been demonstrated in a heterologous system.

These discoveries were made as a result of utilizing a strategy for isolation of the genes which included:

a.) the development of cDNA libraries from plant cells that were treated and plant cells not treated with ABA;

b.) identification of DNA sequences that are specifically induced in the presence of ABA;

c.) identifying those induced sequences that have characteristics of P450 monooxygenases,

d.) correlating the expression pattern of those induced sequences with the structure of the encoded proteins encoded by said sequences to identify a P450 monooxygenase enzyme induced by ABA and,

e.) expressing said enzyme in a heterologous system or as part of a heterologous DNA construct to demonstrate ABA hydroxylase activity.

As described herein, methods for the identification of nucleic acid sequences likely to encode ABA hydroxylase activity are provided. In this method differential screening is used to identify a subset of sequences induced by ABA within a cell population exposed to ABA. Within this subset of sequences that are induced and isolated by standard procedures, P450 monooxygenase sequences are identified using characteristics of the sequences known to be conserved in P450 enzymes.

The enzyme activity responsible for the hydroxylation, and hence inactivation of ABA is known to be a cytochrome P450 monooxygenase (see Krochko et al., 1998, Plant Physiol 118: 849-860) This evidence proved that ABA hydroxylase activity can be induced in Black Mexican Sweet corn suspension cells and is a microsomal enzyme. The biochemical characterization of ABA hydroxylase demonstrates the enzyme is a member of the P450 monooxygenase family. ABA hydroxylase requires NADPH and O₂, the enzyme activity is inhibited by CO, tetcyclacis and oxidized cytochrome c and the CO inhibition is relieved by blue-light, all of these criteria showing that ABA hydroxylase is a cytochrome P450 monooxygenase as these indicators are diagnostic for P450 monooxygenase activity. The induction of ABA hydroxylase activity is inhibited by cycloheximide (a protein synthesis inhibitor) and cordecypin (a RNA synthesis inhibitor) thus indicating the hydroxylase activity is induced at the genetic level following exposure to ABA.

Thus, biochemical characterization of enzyme requirements, appearance and overall enzyme characteristics indicate that the ABA hydroxylase activity responsible for the hydroxylation of ABA is a member of the P450 monooxygenase family. There are a significant number of P450 genes that have been identified by sequencing however, the function of the majority of these sequences remains unknown.

Thus, the present inventors searched for a polynucleotide that can be induced by ABA in corn suspension cultures and encoded structural features of a protein that indicate it is part of the P450 monooxygenase family.

The present invention allowed the inventors to ascribe function to a subfamily of the larger P450 monooxygenase family. A cDNA sequence was isolated from corn suspension cultures that was found to encode a protein that possessed characteristics consistent with an ABA hydroxylase. The partial cDNA clone pJK6-29 (SEQ ID NO:1) was found to be induced by ABA and comprised a protein with P450 monooxygenase motifs. A full-length cDNA clone (SEQ ID NO:2) was isolated and used for searching for other related sequences.

Related sequences were identified within the CYP72A P450 monooxygenase subfamily of Arabidopsis. Two of these sequences (SEQ ID NO:3 and SEQ ID NO:4) were expressed in a heterologous system and ABA hydroxylase activity was demonstrated for one of these sequences. Thus, the inventors have succeeded in identifying a ABA hydroxylase activity and corresponding gene from two plant species, a monocot and a dicot species. The inventors also succeeded in ascribing ABA hydroxylase activity to a member of a gene family where function was previously unknown.

Sequence similarity and conservation of certain amino acid motifs within this subfamily suggest that there are numerous ABA hydroxylase genes within the CYP72A subfamily as supported by biochemical and physiological studies in the art. Examination of the expression pattern of many of these specific sequences indicate the expression patterns are consistent with catabolism of ABA. Thus, it is fully believed that the present invention ascribes function to a subfamily of the P450 monooxygenase family previously unknown in function, and this demonstrated function is the hydroxylation of ABA. Accordingly the present invention provides a function and utility to DNA sequences heretofore described in primary structure but having no assigned functionality.

The present invention encompasses, in one embodiment, a nucleic acid pBE10-30-3 encoding an enzyme from corn with ABA hydroxylation activity and similarity to the general family of P450 monooxygenase enzymes from plants. The specification also describes the partial cDNA pJK6-29 sequence used to derive the full-length pBE10-30-3 cDNA. Based on the structure and expression of this coding sequence, and on the biochemical and genetic evidence, it has been designated as ABA 8′-hydroxylase. The gene encoding this enzyme activity has not previously been identified in the art. Recently similar genes have been isolated (Persans M., et al., 2001, Plant Physiol 125: 1126-1138, Irmler S., et al., 2000, Plant Journal 24: 797-804), however, the role of these genes in ABA metabolism was not described.

The present invention also encompasses, in one embodiment, a series of genes from the CYP72A subfamily of the super family of P450 monooxygenase genes. Genes from Arabidopsis thaliana have been isolated and shown to possess enzymatic activity capable of specifically hydroxylating ABA in a heterologous system. One of these genes, CYP72A7 has been shown to possess a 7′-hydroxylase activity in a heterologous yeast system. Thus, the present invention provides clear evidence of the nature of ABA hydroxylase genes and functional expression thereof.

The identification of this unique genetic activity allows for development of novel strategies to manipulate the catabolism of ABA in plant cells. The novel nucleic acid can also be used to identify species-specific members of this plant gene family, and to utilize those sequences for alteration of ABA catabolism. In addition to the use of the pBE10-30-3 sequence for the genetic modification of plant tissue, the sequence can also be used to isolate corresponding related similar or identical sequences from other plant species.

In particular, the identification of an ABA 7′-hydroxylase activity allows for the identification of an ABA 8′-hydroxylase activity through sequence comparison. This can be conducted within a plant species or between plant species. Similar or identical sequences can be isolated. It has been discovered that the CYP72A P450 monooxygenase gene subfamily encodes a small number of enzymes capable of hydroxylation of ABA. The in planta function of the enzymes encoded by the CYP72A family was heretofore unknown, and the information described in the present invention demonstrates that this gene family encodes enzymes capable of hydroxylating ABA.

It has been shown by the present inventors that an ABA-induced genetic activity found in corn suspension cultures treated with ABA encodes a P450 monooxygenase enzyme. The sequence of this enzyme was used to identify related sequences from Arabidopsis. The inventors utilized one of these related sequences for heterologous expression in a yeast system and demonstrated that the enzyme encoded an ABA 7′-hydroxylase activity. It is fully expected that other related sequences described will also encode ABA hydroxylase activities.

The present invention encompasses the nucleotide and peptide sequences disclosed in the present application, as well as other DNA sequences that show a degree of homology to the sequences disclosed herein. For this purpose, such homologous sequences may be identified by any one of a number of routine techniques that are known in the art for the isolation and characterization of homologous DNA or peptide sequences from species that are either the same or different from the original species.

As described, the similarity or identity of two polypeptide or polynucleotide sequences is determined by comparing sequences. In the present invention, sequence comparisons were conducted using the BLAST programs, three designed for nucleic acid sequences, (BLASTN, BLASTX and TBLASTX) and two designed for protein sequences (BLASTP and TBLASTN) (Coulson A., 1994, Trends in Biotechnology, 12:76-80). The BLASTX program is publicly available from NCBI and other sources such as the BLAST Manual, Altschul, S., et al., NCBI NLM NIH Bethesda Md. 20984, also http://www.ncbi.nlm.nih.gov/BLAST/blast_help.html provides online help and further literature references for BLAST and related protein analysis methods, and Altschul S., et al., 1990, J Mol Biol 215:403-410.

The 333 bp cDNA clone pJK6-29 of corn P450 cDNA isolated by subtractive hybridization shows strong homologies to cytochrome P450 genes of the CYP72A gene subfamily. Comparison of the sequence of JK6-29 with deposited sequences in gene databases using the BLAST search programs and the default setting reveals similarity with a subset of P450 monooxygenase clones identified as the CYP72A gene subfamily.

Members of the CYP72A gene family was one of the first P450 monooxygenase genes to be cloned in plants and was originally cloned from periwinkle (Vetter H.-P., et al., 1992, Plant Physiol 100: 998-1007). Those authors tentatively assigned geraniol-10-hydroxylase activity to this protein based on induction kinetics, however, they were unable to demonstrate conclusively that this P450 had GE10H activity. Heterologous expression of these P450s in tobacco failed to confirm the GE10H activity (Mangold U., et al., 1994, Plant Science 96: 129-136) although since that time there have been many citations in the literature referencing that paper in describing the function of the CYP72A genes. None of these functions have been clearly demonstrated, making the activity of the enzymes encoded for by the CYP72A gene family unknown. Moreover, a patent for plant geraniol/nerol 10-hydroxylase activity has demonstrated the GE10H activity to be encoded by CYP76C1 gene sequence (U.S. Pat. No. 5,753,507), thus the function of the CYP72A gene family remained unknown until the present invention. However, CYP72As are still frequently described in the literature and even in some newer databases as possible geraniol 10-hydroxylases (see Stanford Microarray Database). Now, with the discovery of JK6-29 and the isolation of the full-length sequence pBE10-30-3, the function of CYP72A sequences is better understood to comprise at least ABA hydroxylation enzymes, including ABA 7′-hydroxylase as demonstrated by in vitro enzyme activity.

Organization of the CYP72A Gene Subfamily in Plants

The CYP72A gene family has been found in most plant species including Arabidopsis, rice, tomato, potato, corn, Medicago (alfalfa), soybean, sorghum and barley. For the most part, the assignment of a DNA sequence to the CYP72A subfamily has been based on sequence comparison and a high degree of similarity within these various sequences. A combination of genomic DNA sequences as well as EST sequences have provided information on the occurrence of these gene sequences. The family has not however, been characterized in regard to function.

In the present invention, in at least one embodiment, homologous sequences from Arabidopsis were identified and the related genes were assigned to a moderate size (9 members) cytochrome P450 gene subfamily whose members are arrayed in tandem on chromosome 3. This sequence information was first submitted to GenBank by the Kazusa DNA Research Institute on Feb. 1, 1999 (P1 clone MIE1; GenBank AB023038, Sato et al., DNA Research 7:131-135, 2000), but the function of these genes was unknown.

The members of the CYP72A gene family in Arabidopsis are found in Genbank and are listed as follows:

| CYP72A7 | AB023038, chromosome 3, MIE1 40525- | protein id: |
|---|---|---|
| | 42463 | BAB02393.1 |
| CYP72A8 | AB023038, chromosome 3, MIE1 42938- | protein id: |
| | 44813 | BAB02394.1 |
| CYP 72A9 | AB023038, chromosome 3, MIE1 45293- | protein id: |
| | 47369 | BAB02395.1 |
| CYP 72A10 | AB023038, chromosome 3, MIE1 47816- | protein id: |
| | 49747 | BAB02396.1 |
| CYP 72A11 | AB023038, chromosome 3, MIE1 50166- | protein id: |
| | 52125 | BAB02397.1 |
| CYP 72A12P | AB023038, chromosome 3, MIE1 52192- | |
| (exon 5 only) | 52485 | |
| CYP 72A13 | AB023038, chromosome 3, MIE1 52920- | protein id: |
| | 54871 | BAB02398.1 |
| CYP 72A14 | AB023038, chromosome 3, MIE1 62438- | protein id: |
| | 64422 | BAB02400.1 |
| CYP 72A15 | AB023038, chromosome 3, MIE1 65370- | protein id: |
| | 67270 | BAB02401.1 |

The sequence identity of these nine family members ranges between 6392% at the DNA level, while the protein sequence identity range between 55-90%. Of interest to note is that the sequence identity to the corn full-length cDNA (SEQ ID NO:2) isolated by the present inventors ranges from 50-60% at the DNA level and 50-55% at the level of the protein sequence. This is illustrated by the comparative table shown below, where eight members of the Arabidopsis CYP72A gene family are compared to each other as well as compared to the corn cDNA clone (SEQ ID NO:2).

The alignment of the sequences was based on ClustalW (1.81) analysis of corn sequence (cDNA and deduced protein) and the 8 full-length Arabidopsis genes as described above. In these tables, each of the CYP72A genes identified are abbreviated for convenience by deletion of the internal “72A” notation in the sequence name. Thus, CYP72A7 is referred to as CYP7 for this analysis.

TABLE 1
Comparison of the Arabidopsis CYP72A full length cDNA sequences

| cDNA | CYP7 | CYP8 | CYP9 | CYP10 | CYP11 | CYP13 | CYP14 | CYP15 | corn |
|---|---|---|---|---|---|---|---|---|---|
| CYP7 | 100 | | | | | | | | |
| CYP8 | 66 | 100 | | | | | | | |
| CYP9 | 73 | 63 | 100 | | | | | | |
| CYP10 | 76 | 65 | 77 | 100 | | | | | |
| CYP11 | 74 | 66 | 77 | 87 | 100 | | | | |
| CYP13 | 74 | 67 | 78 | 87 | 92 | 100 | | | |
| CYP14 | 74 | 65 | 77 | 85 | 87 | 87 | 100 | | |
| CYP15 | 73 | 64 | 77 | 87 | 90 | 88 | 86 | 100 | |
| corn | 53 | 50 | 52 | 59 | 60 | 50 | 60 | 60 | 100 |

As evidenced by this table, the gene sequences of this family are highly similar and the identity ranges from 63-92%, with a typical identity being about 65-70% for the least related, 74-78% for the more related sequences and 85-92% for the most related sequences. Nearly all of the Arabidopsis sequences of the CYP72A family show at least 50% identity, with some specific sequences showing 60% similarity.

This similarity extends to the protein level, where the translated protein sequences show similar ranges of identity as shown below.

TABLE 2
Comparison of the Arabidopsis CYP72A deduced protein sequences

| protein | CYP7 | CYP8 | CYP9 | CYP10 | CYP11 | CYP13 | CYP14 | CYP15 | corn |
|---|---|---|---|---|---|---|---|---|---|
| CTP7 | 100 | | | | | | | | |
| CYP8 | 58 | 100 | | | | | | | |
| CYP9 | 67 | 55 | 100 | | | | | | |
| CYP10 | 72 | 56 | 69 | 100 | | | | | |
| CYP11 | 71 | 57 | 70 | 83 | 100 | | | | |
| CYP13 | 72 | 58 | 71 | 84 | 90 | 100 | | | |
| CYP14 | 68 | 57 | 70 | 79 | 83 | 83 | 100 | | |
| CYP15 | 70 | 56 | 71 | 83 | 87 | 88 | 83 | 100 | |
| corn | 51 | 50 | 53 | 53 | 54 | 53 | 54 | 55 | 100 |

It is clear that the members of this CYP72A subfamily are highly similar. When members of this family are compared to P450 monooxygenase genes in general, the similarity is not evident. For example, comparison of Arabidopsis CYP72A7 sequences with other members of the P450 monooxygenase genes from plants reveal that the CYP72A family can be discriminated from other P450 monooxygenases by a difference of approximately at least 10% in homology between the CYP72A types and other CYP P450 monooxygenases. Typically CYP72A genes exhibit at least 50% identity to each other, whereas non CYP72A genes exhibit less that 40% identity to CYP72A genes. It is the CYP72A genes that have demonstrated ABA hydroxylase activity as described within the present invention.

For example, a BLASTP 2.2.1 analysis of protein encoded by the Arabidopsis CYP72A7 (SEQ ID NO:5) compared to other P450 monooxygenase genes found at the NCBI database, (default settings, sequences shown in order of identity) reveal the following homologies. In this analysis, “identity” refers to identical amino acids, whereas “positives” refer to amino acids that are identical or conservatively substituted.

BLASTP 2.2.1 analysis of deduced amino acid
sequence of Arabidopsis CYP72A7 (SEQ ID NO: 5)

| Sequenced compared to | “identity” and “positives” score |
|---|---|
| CYP72A7 | 100% |
| CYP72A13 | 74% identity, 85% positives |
| CYP72A11 | 73% identity, 83% positives |
| CYP72A15 | 71% identity, 83% positives |
| CYP72A10 | 73% identity, 83% positives |
| CYP72A9 | 69% identity, 82% positives |
| CYP72A14 | 70% identity, 81% positives |
| CYP72A8 | 58% identity, 77% positives |
| CYP72A18 (Oryza sativa) | 55% identity, 70% positives |
| CYP72A1 (Catharanthus roseus) | 51% identity, 71% positives |
| | (L10081) |
| CYP72A-like (seq B, C. roseus) | 50% identity, 70% positives |
| | (L19074) |
| Lolium-62 | 52% identity, 68% positives |
| Lolium-78 | 52% identity, 68% positives |
| CYP72A-like (seq C, C. roseus) | 50% identity, 70% positives |
| | (L19075) |
| CYP72A20 (O. sativa) | 49% identity, 64% positives |
| Lolium-83 | 52% identity, 69% positives |
| Lolium-22 | 52% identity, 69% positives |
| CYP72A-like (O. sativa) | 50% identity, 69% positives |
| | (AP002899) |
| Lolium-79 | 52% identity, 69% positives |
| CYP72A24 | 53% identity, 69% positives |
| CYP72A17 | 49% identity, 67% positives |
| CYP72A23 | 51% identity, 68% positives |
| CYP72A19 | 50% identity, 66% positives |
| CYO72A21 | 50% identity, 66% positives |
| CYP72A22 | 49% identity, 66% positives |
| CYP72A-like (tobacco) | 49% identity, 67% positives |
| | (U35226) |
| CYP72A-like (tomato) | 47% identity, 65% positives |
| | (AF249329) |
| CYP72A25 (O. sativa) | 44% identity, 61% positives |
| Non-CYP72A genes | |
| CYP72B1 (Arabidopsis) | 40% identity, 62% positives |
| CYP72C1 (Arabidopsis) | 41% identity, 61% positives |
| CYP709B2 (Arabidopsis) | 39% identity, 57% positives |
| CYP709B3 (Arabidopsis) | 36% identity, 56% positives |
| CYP709B-like (maize) | 37% identity, 55% positives |
| CYP721A1 (Arabidopsis) | 38% identity, 56% positives |
| CYP709B1 (Arabidopsis) | 38% identity, 58% positives |
| CYP709B-like (O. sativa) | 36% identity, 54% positives |
| CYP709A2 (Arabidopsis) | 36% identity, 56% positives |
| CYP709A1 (Arabidopsis) | 36% identity, 55% positives |
| CYP714A-like (Lolium) | 31% identity, 51% positives |
| CYP714A1 (Arabidopsis) | 33% identity, 51% positives |
| CYP714A2 (Arabidopsis) | 32% identity, 52% positives |
| CYP715A1 (Arabidopsis) | 29% identity, 50% positives |
| CYP715A-like (Persea americana) | 34% identity, 58% positives |
| CYP709B-like (O. sativa) | 37% identity, 57% positives |
| CYP4F2 (Homo sapiens) | 28% identity, 46% positives |
| CYP709-like (Agrobacterium | 30% identity, 47% positives |
| tumefaciens) | |

Thus, it is apparent that a minimal sequence identity of approximately 50% is required for a new gene to be identified as a member of the CYP72A family and hence be a candidate for ABA hydroxylase activity.

This similarity extends to other P450 monooxygenase genes that are predicted to possess ABA hydroxylase activity. For example, when the deduced amino acid sequence of the pBE10-30-3 cDNA (SEQ ID NO:2) is used in a BLASTP 2.2.1 with P450s in general, a clear discrimination between CYP72A and other P450 monooxygenase genes is observed. This is shown below. As above, in this analysis, “identity” refers to identical amino acids, whereas “positives” refer to amino acids that are identical or conservatively substituted.

BLASTP 2.2.1 comparison of deduced amino acid
sequence of pBE10-30-3 cDNA (SEQ ID NO: 2)

| Sequenced compared to | “identity” and “positives” score |
|---|---|
| Lol-78 | 80% identity, 90% positives |
| Lol-62 | 79% identity, 89% positives |
| CYP72A18 | 77% identity, 88% positives |
| Lol-83 | 79% identity, 89% positives |
| Lol-79 | 79% identity, 89% positives |
| Lol-22 | 78% identity, 88% positives |
| CYP72A-like (Oryza sativa) | 69% identity, 81% positives |
| | (AP002899) |
| CYP72A23 (O. sativa) | 65% identity, 77% positives |
| CYP72A21 (O. sativa) | 64% identity, 77% positives |
| CYP72A19 (O. sativa) | 63% identity, 77% positives |
| CYP72A22 (O. sativa) | 63% identity, 76% positives |
| CYP72A20 (O. sativa) | 61% identity, 74% positives |
| CYP72A17 (O. sativa) | 61% identity, 75% positives |
| CYP72A24 (O. sativa) | 62% identity, 75% positives |
| CYP72A15 (Arabidopsis) | 57% identity, 72% positives |
| CYP72A14 (Arabidopsis) | 55% identity, 70% positives |
| CYP72A10 (Arabidopsis) | 55% identity, 70% positives |
| CYP72A11 (Arabidopsis) | 55% identity, 70% positives |
| CYP72A25 (O. sativa) | 53% identity, 68% positives |
| CYP72A13 (Arabidopsis) | 55% identity, 70% positives |
| CYP72A9 (Arabidopsis) | 54% identity, 68% positives |
| CYP72A7 (Arabidopsis) | 53% identity, 69% positives |
| CYP72A8 | 52% identity, 70% positives |
| CYP72A1 (Catharanthus roseus) | 49% identity, 68% positives |
| | (L10081) |
| CYP72A-like (seq B, C. roseus) | 49% identity, 69% positives |
| | (L19074) |
| CYP72A-like (seq C, C. roseus) | 49% identity, 68% positives |
| | (L19075) |
| CYP72A-like (tobacco) | 50% identity, 66% positives |
| | (U35226) |
| CYP72A-like (tomato) | 46% identity, 63% positives |
| | (AF249329) |
| Non-CYP72A genes | |
| CYP72B1 (Arabidopsis) | 43% identity, 62% positives |
| CYP72C1 (Arabidopsis) | 41% identity, 61% positives |
| CYP709B-like (O. sativa) | 42% identity, 58% positives |
| CYP709B-like (maize) | 39% identity, 56% positives |
| CYP709B2 (Arabidopsis) | 39% identity, 57% positives |
| CYP709B1 (Arabidopsis) | 38% identity, 57% positives |
| CYP721A1 (Arabidopsis) | 40% identity, 58% positives |
| CYP709B3 (Arabidopsis) | 39% identity, 57% positives |
| CYP709A1 (Arabidopsis) | 37% identity, 59% positives |
| CYP714A2 (Arabidopsis) | 32% identity, 56% positives |
| CYP714A1 (Arabidopsis) | 34% identity, 54% positives |
| CYP709A2 (Arabidopsis) | 36% identity, 57% positives |
| CYP714A-like (Lolium) | 33% identity, 53% positives |
| CYP715A1 (Arabidopsis) | 30% identity, 50% positives |
| CYP715A-like (Persea americana) | 34% identity, 56% positives |
| CYP709B-like (O. sativa) | 40% identity, 59% positives |
| CYP709-like (Agrobacterium | 30% identity, 45% positives |
| tumefaciens) | |

Accordingly, it is clear that it is possible to distinguish the CYP72A family of genes from P450 monooxygenase genes in general by overall sequence similarity. It is also noted that there are specific motifs within the CYP72A family that distinguishes this family from other P450 monooxygenases.

There are several conserved regions that have become apparent from multi-sequence alignments of putative ABA hydroxylase genes (CYP72As from different species identified by BLAST and homology analysis). These include:

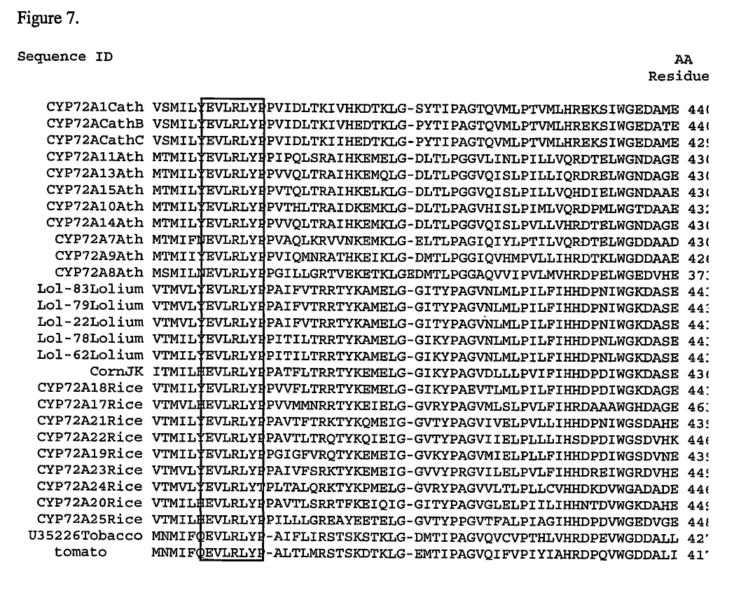

A 6 amino acid sequence ‘EVLRLY’ (SEQ ID NO.22) that is conserved in the CYP72A (8 genes) and the CYP72C (I gene) P450 subfamilies in Arabidopsis. This sequence is found in only one other P450 in Arabidopsis (CYP715A1) and only one non-P450 protein sequence in Arabidopsis (BAB01891.1). This small sequence is positioned approximately 80 amino acids upstream of the Cysteine of the heme-binding region. This sequence is also found in all of the other CYP72A genes identified from other plant species. See FIG. 7.

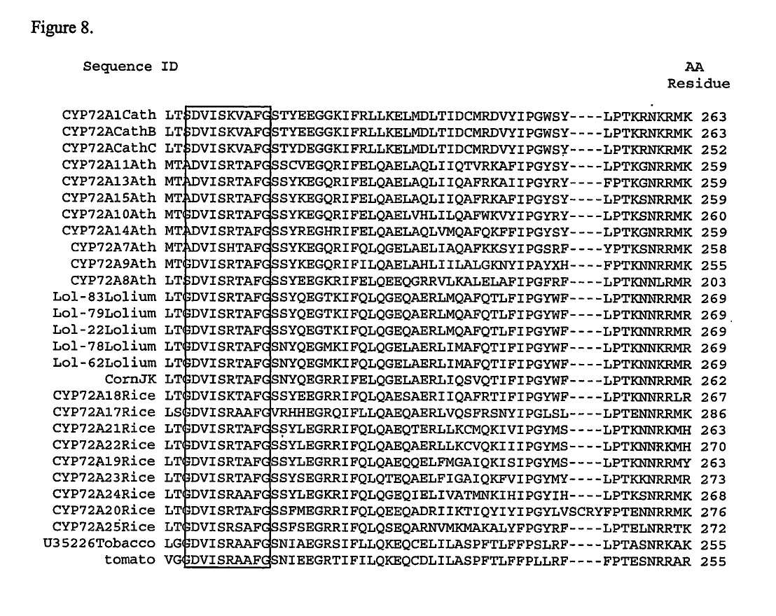

A second conserved region also occurs in the CYP72As. A 9 amino acid sequence with the highly conserved elements: “DVIS[KRH]xAFG” (SEQ ID NO.23), is found approximately 240-250 amino acids upstream of the conserved Cysteine of the heme-binding region. In Arabidopsis this sequence is specific for the CYP72A and CYP72B subfamilies. See FIG. 8.

Thus, only the CYP72A subfamily in Arabidopsis has both of these sequences and this forms a basis for selecting CYP72A genes in general that encode ABA hydroxylase activity.

Thus, in addition to overall sequence homologies, there are conserved identical amino acid sequences in CYP72A plant genes known to possess ABA hydroxylase activity as described above. These sequences provide another means to specifically identify genes encoding an ABA hydroxylase activity.

A third region of conserved amino acid residues includes eight amino acids located approximately 170 amino acids upstream of the Cysteine residue of the heme-binding region. The conserved sequence “D[LI]LG[LIV][LM][VL][EQK]” (SEQ ID NO.24) is generally found in CYP72A genes and constitutes a third region of amino acid similarity that is diagnostic for CYP72A genes that can encode ABA hydroxylase activity. CYP72C1 does not show conservation in this region. The only other Arabidopsis protein that has been shown to contain this sequence is a P450 monooxygenase named CYP709B1, which is not a member of the CYP72A subfamily. Notably, CYP709B1 does not have the other conserved amino acid sequences and hence would not be a candidate for ABA hydroxylase activity. Other P450 monooxygenase genes that have related sequences include CYP72B1, which has a conservative substitution of ‘I’ in the 7th position ([VLI]).

Thus, the combination of sequence identity across the entire encoded protein, as well as the presence of these highly conserved sequence motifs provide key features of the P450 monooxygenase genes that are candidates for ABA hydroxylase activity. As described within the present invention, one of these CYP72A genes was demonstrated by biochemical conversion, to possess an ABA 7′-hydroxylase activity.

Another plant species that contains a small number of CYP72A related sequences is rice, (Oryza sativa), which also has 9 CYP72A genes, similar to that seen for Arabidopsis. (http://drnelson.utmem.edu/rice.cyp72 as.html). The designation of the rice CYP72A genes which are clustered on chromosome 1 are as follows:

CYP72A17 AP002839 Oryza sativa genomic DNA, chromosome 1, 36553-39431

CYP72A18 AP002839 Oryza sativa genomic DNA, chromosome 1, 41630-44993

CYP72A19 AP002839 Oryza sativa genomic DNA, chromosome 1, 51708-53699

CYP72A20 AP002839 Oryza sativa genomic DNA, chromosome 1, 56581-59004

CYP72A21 AP002839 Oryza sativa genomic DNA, chromosome 1, 61972-63890

CYP72A22 AP002839 Oryza sativa genomic DNA, chromosome 1, 66435-68424

CYP72A23 AP002839 Oryza sativa genomic DNA, chromosome 1, 72149-74091

CYP72A24 AP002839 Oryza sativa genomic DNA, chromosome 1, 109964-113787

CYP72A25 AP002839 Oryza sativa genomic DNA, chromosome 1, 115472-117492

In analyzing sequence similarities, deduced amino acid sequences are used more frequently than cDNA sequences in order to focus on structural features of the derived protein.

The similarity of the rice CYP72A deduced protein sequences to the Arabidopsis CYP72A protein sequences ranges from 46-55%. This range of similarity is typical of the range of similarity already identified between the Arabidopsis gene family and the comparison of that gene family to SEQ ID NO:2. Thus it is fully believed that the rice CYP72A gene family described above represents genes encoding an ABA hydroxylase activity.

These comparisons within the CYP72A gene family can also be extended to other species where little is known as to the nature of P450 monooxygenase genes, for example, a number of Lolium rigidum genes have been isolated by a PCR strategy (these have not been assigned CYP numbers as of yet) and these sequences show a high level of similarity to the CYP72A gene family (http://drnelson.utmem.edu/lolium.html, and Fischer T. C., Klattig J. T. and Gierl A. A general cloning strategy of divergent plant cytochrome P450 genes and its application in Lolium rigidum and Ocimum basilicum. Unpublished).

Comparison of the Rice CYP72A sequences to the deposited sequences from Lolium listed below demonstrate striking similarities, indicating their likely function to include an ABA hydroxylase. These similarities include the conserved amino acids regions as described (see FIGS. 7 and 8). These sequences are listed below.

Lolium rigidum clone Lol-83 AF321870 78% to 72A18 (rice)

Lolium rigidum clone Lol-79 AF321869 79% to 72A18 (rice)

Lolium rigidum clone Lol-78 AF321868 78% to 72A18 (rice)

Lolium rigidum clone Lol-62 AF321867 77% to 72A 18 (rice)

Lolium rigidum clone Lol-22 AF321866 78% to 72A 18 (rice)

It should be noted that the clone Lol-83 AF321870 has 54% similarity to the Arabidopsis CYP72A15 clone. Thus, members of the CYP72A family in plants that are associated with ABA hydroxylase activity are typically found to have between 50 and 80% similarity at the protein level between unrelated plant species. Within a plant species that similarity can be as high as 92%. Even between monocots and dicots the similarity of the coding regions does not fall below 50%.

It has been found that the similarity of CYP72A genes is found among diverse species. Using the deduced protein sequence of Arabidopsis CYP72A14 and the deduced protein sequence of pBE10-30-3 cDNA (SEQ ID NO:2), a TBLASTN similarity search of the TIGR Gene Indices database (http://www.tigr.org/tdb/tgi.shtml) was conducted and the results reveal that many CYP72A sequences previously identified can now be assigned a potential function in ABA catabolism. Representative CYP72A sequences identified in this database include:

Tomato:

TIGR contig TC85158 (26 ESTs)

TIGR contig TC94028 (47 ESTs)

TIGR contig TC93825 (45 ESTs)

TIGR contig TC85178 (29 ESTs)

Potato:

TIGR contig TC19836 (43 ESTs)

TIGR contig TC19818 (53 ESTs)

Medicago trunculata:

TIGR contig TC31893 (33 ESTs)

TIGR contig TC32102 (44 ESTs)

TIGR contig TC28776 (4 ESTs)

TIGR contig TC28751 (16 ESTs)

Soybean:

TIGR contig TC73251 (36 ESTS)

TIGR contig TC63297

Sorghum

TIGR contig TC20554 (18 ESTS)

Barley

TIGR contig TC3433 (2 ESTs)

Maize

TIGR contig TC76645 (2 ESTS)

TIGR contig TC76455 (3 ESTs)

Rice

TIGR contig TC52700 (12 ESTs)

TIGR contig TC54931 (1 EST)

TIGR contig TC53498 (1 EST)

TIGR contig TC50219 (1 EST)

Screening these putative sequences for the presence of the conserved amino acid sequences, “EVLRLY” (SEQ ID NO.22), “DVIS[KRH]xAFG” (SEQ ID NO.23) and “D[LI]LG[LIV][LM][VL][EQK]” (SEQ ID NO.24) as described, also allows for the identification of CYP72A genes likely to contain ABA hydroxylase activity as these sequences are conserved in these proteins.

Thus, it is possible to identify a large number of CYP72A sequences that are highly similar to CYP72A sequences both at the overall protein identity as well as at the level of specific conserved sequences to identify genes that encode an ABA hydroxylase activity. Identification of these sequences allows the isolation of an ABA hydroxylase gene by routine procedures such as identification of specific ESTs or sequences that are induced or expressed in response to ABA related physiological changes, or by matching the expression pattern of these sequences to expression patterns that are typical of ABA related genes.

Expression of the CYP72A Gene Subfamily:

As one aspect of the present invention, identification of CYP72A sequences that are associated with physiological events associated with ABA provides an important element for screening within the CYP72A family for functional genes and genes most likely to encode an ABA hydroxylase activity. ABA-hydroxylase is found predominantly in dividing and meristematic tissues, as well as leaves. There is reduced expression in the dark. Typically ABA hydroxylase is up-regulated in leaves relative to flowers, down-regulated in iron deficient plants, up-regulated under conditions of high CO₂, up-regulated in some mutants and down-regulated in suspension cells relative to leaves. A search of Genbank was conducted to generate a list of Arabidopsis CYP72A ESTs; a total of 27 EST records were found. The pattern of expression associated with these EST sequences were analyzed and the following conclusion were drawn that describe the general expression patterns of CYP72A genes in Arabidopsis: individual genes are not limited in their expression to particular tissues; CYP72A9 and CYP72A13 are the only members of this gene family so far found in developing seed tissues; CYP72A8 has so far only been isolated from roots; CYP72A 10 has not been associated with any ESTs. It is believed that the genes most likely to be involved in ABA catabolism in planta are CYP15, CYP14, CYP13 and CYP11 based on the EST prevalence data and expression analysis of these cDNAs using microarrays (StanfordMicroarrayDatabase).

Demonstration of ABA Hydroxylase Activity Associated with CYP72A Genes.

Several P450 ESTs belonging to this P450 CYP72A subfamily of Arabidopsis were obtained from ABRC (Arabidopsis Biological Resource Center) and the inserts were sequenced and the sizes determined. These P450s included full-length sequences for CYP72A7 and CYP72A14. These cDNAs were expressed in a yeast expression system and ABA 7′-hydroxylase activity was demonstrated in the cultures in which the CYP72A7 gene was inserted. These yeast expression results using the Arabidopsis CYP72A7 gene show that (+)-ABA fits into the substrate access channel in this P450 subfamily and that one of the methyl groups of the substrate is appropriately positioned for hydroxylation. (+)-ABA 7′-hydroxylase activity is found naturally in plants and this observed activity in yeast may represent an allelic variant within this gene family.

The data outlined above provide compelling evidence that the ABA 7′- and 8′-hydroxylases are coded by the CYP72A cytochrome P450 subfamily in plants. This is a new discovery of cytochrome P450 gene function, this function was not known previously. Indeed, one of the members of the CYP72A family was demonstrated to encode an activity capable of hydroxylating ABA at the 7′ position.

The present inventors have accumulated a substantial set of data that demonstrates that the newly discovered polynucleotides encode ABA hydroxylase enzymes. The supporting data includes: isolation of a P450 cDNA (pJK6-29, and subsequently the full-length corresponding cDNA pBE10-30-3) from the ABA-induced subtracted maize cDNA library; confirmation by Northern expression analysis that this P450 coding sequence is induced by ABA; the time-course of that induction is consistent with this P450 being ABA 8′-hydroxylase; isolation of a full-length maize cDNA that allows for expression of enzyme activity in plant cells; isolation of homologous ABA hydroxylase sequences from other plant species and; demonstration in yeast of (+)-ABA 7′-hydroxylase activity with the homologous Arabidopsis CYP72A7 sequence.

Thus with the JK6-29 cDNA and pBE10-30-3 sequences it is possible to isolate polynucleotides encoding ABA hydroxylase activity. These polynucleotides may be used to alter ABA catabolism in plant cells.

The use of gene inhibition technologies such as antisense RNA (e.g., U.S. Pat. No. 4,801,540, U.S. Pat. No. 5,107,065; U.S. Pat. No. 5,453,566, use of ribozymes (e.g., U.S. Pat. No. 4,987,071; U.S. Pat. No. 5,037,746; U.S. Pat. No. 5,116,742; U.S. Pat. No. 5,354,855) or co-suppression (e.g., U.S. Pat. No. 5,034,323; 1994, U.S. Pat. No. 5,283,184, & U.S. Pat. No. 5,231,020) or double stranded RNA interference (Chuang C.-F. and Meyerowitz E. M., 2000, PNAS 97: 4985-4990, Smith N. A., et al., 2000, Nature 407: 319-320) is contemplated within the scope of the present invention. In these approaches, the isolated ABA hydroxylase gene sequence is operably linked to a suitable regulatory element. The chimeric gene is introduced into a plant cell and a plant cell recovered wherein said gene is integrated into the plant chromosome. The plant cell is induced to regenerate and a whole plant is recovered. The use of these techniques has been well-described in the art, and it is apparent that polynucleotide sequences encoding ABA hydroxylase enzymes, or portions thereof can be employed within the scope of the present invention to alter ABA catabolism.

For example, inhibition of the expression of an ABA 8′-hydroxylase may provide a number of beneficial characteristics to plants. These may include: increased seed dormancy to allow fall planting of certain crops, reduced stomatal conductance to reduce water loss, reduced growth rate in some crops to better control production, reduced shoot growth versus root growth to allow for greater establishment of a crop, larger seeds because of more storage product accumulation and in general greater stress tolerance.

In addition, it is contemplated that over-expression of ABA hydroxylation enzymes can be used as a mechanism for altering ABA levels. This method may employ the use of the coding sequence, under the control of an appropriate promoter, to express ABA hydroxylation activity in a temporal and spatial fashion not normally found in plants. The promoter may be constitutive, tissue specific or inducible.

Expected results of over-expression of ABA 8′-hydroxylase in plants leading to ABA deficient plants include: seeds that may germinate faster after collection, seeds may be non-dormant whereas before they might have been dormant, increased branching, changes in fertility, reduced plant stature and leaf area (dwarf plants), reduced root growth versus shoot growth to provide more biomass.

The method further relies on the use of transformation to introduce the gene encoding the enzyme into plant cells. Transformation of the plant cell can be accomplished by a variety of different means. Methods that have general utility include Agrobacterium based systems, using either binary and cointegrate plasmids of both A. tumifaciens and A. rhyzogenies. (e.g., U.S. Pat. No. 4,940,838, U.S. Pat. No. 5,464,763), the biolistic approach (e.g, U.S. Pat. No. 4,945,050, U.S. Pat. No. 5,015,580, U.S. Pat. No. 5,149,655), microinjection, (e.g., U.S. Pat. No. 4,743,548), direct DNA uptake by protoplasts, (e.g., U.S. Pat. No. 5,231,019, U.S. Pat. No. 5,453,367) or needle-like whiskers (e.g., U.S. Pat. No. 5,302,523). The disclosures of these references are specifically incorporated herein by reference. Any method for the introduction of foreign DNA and/or genetic transformation of a plant cell may be used within the context of the present invention.

Accordingly, in a preferred embodiment of the invention the subject method includes a method for modifying the catabolism of ABA in a plant comprising:

(a) Introducing into a plant cell capable of being transformed and regenerated to a whole plant a genetic construct comprising a first DNA expression cassette that comprises, in addition to the DNA sequences required for transformation and selection in plant cells, a DNA sequence that encodes the polynucleotide encoding a plant ABA hydroxylase sequence, said sequence encoding a ABA 7′ or 8′-hydroxylase activity, operably linked to a suitable transcriptional regulatory region and,

(b) recovery of a plant which contains said recombinant DNA.

It is apparent to the skilled artisan that the polynucleotide encoding the ABA hydroxylase sequence can be in the antisense (for inhibition by antisense RNA) or sense (for inhibition by co-suppression, or over-expression) orientation, relative to the transcriptional regulatory region, or a combination of sense and antisense RNA to induce double stranded RNA interference. A transcriptional regulatory region is often referred to as a promoter region and there are numerous promoters that can be used within the scope of the present invention.

It will be apparent to the skilled practitioner that any number of specific alterations in ABA catabolism will lead to changes in processes and plant physiology controlled by ABA. These include alteration in water loss or transpiration and water use efficiency; responses to pathogens and wounding; embryogenesis, seed development and maturation including seed storage product accumulation (protein, oil), seed dormancy, seedling vigour and seed desiccation tolerance; plant architecture, including root growth and morphology, plant height and root-to-shoot ratios; abiotic stress tolerance; fertility and reproduction; and fruit (silique) maturation, dehiscence and abscission.

Alteration in ABA catabolism by increased or decreased expression of ABA hydroxylase enzymes can include changes in: Abscission, Bud Dormancy, Fruit Ripening, Seed Maturation including the Accumulation of Storage Products, Seed Desiccation Tolerance, Plant Architecture, Fertility, Seed Dormancy, Elongation Growth, Water Loss and Root Growth, and Abiotic Stress Tolerance. Other alterations of plant physiology may also be envisioned by the use of the ABA hydroxylase enzymes and the use of a tissue specific or inducible promoter.

It is also apparent to the skilled practitioner that the exemplified coding sequences can be used to isolate genes from other plant species with similar function. As described, members of the CYP72A gene family have been shown to encode an ABA hydroxylase activity, and there are many highly similar sequences in other plant species that can be easily evaluated for ABA hydroxylase activity as described herein.

Thus, homologous sequences to pBE10-30-3 are P450 monooxygenases that are found associated with stress or developmental conditions known to be modulated by ABA.

These homologous sequences will encode 7′- and 8′-ABA hydroxylases and be part of the P450 CYP72A monooxygenase family. It is further apparent to the skilled artisan that these sequences can be used in heterologous plant species to alter ABA catabolism.

It is further contemplated within the scope of the present invention that the activity of these enzymes may be used directly or be modified to hydroxylate substrates related to ABA. For example, analogs of ABA have been described where the ABA molecule is modified to resist degradation (U.S. Pat. No. 6,004,905). These modified ABA compounds could be used as novel herbicides since application of these compounds would lead to persistent ABA effects, including growth retardation in some cases. Modification of the 7′- and 8′-hydroxylase genes to more effectively degrade these modified ABA compounds would allow those plants comprising a modified ABA hydroxylase gene to grow more efficiently in the presence of the modified ABA analogs than plants without the modified hydroxylase activity. Alternatively, modification of the enzyme to oxidize ABA to produce 9′-hydroxyABA (a non-natural hormone metabolite of unknown biological activity) would permit a further means of manipulating ABA activities.

As one example of an enzyme modification, the gene encoding an enzyme capable of 7′- or 8′-hydroxylation of ABA can be modified and/or selected for increased activity by using such techniques as site-specific modification, mutation and selection of altered specificity in heterologous systems such as bacterial or fungal cells. This may include expression of the gene encoding said altered enzyme in bacterial cells unable to metabolize ABA analogs, selecting bacteria that express the enzyme and are able to hydroxylate the analogs as determined by a variety of analytical techniques including high performance liquid chromatography/mass spectrometry (HPLC/MS).

It is also possible to mutagenize a population of plant cells and select plant cells for resistance to the ABA analogs. In this approach, plant cells or plant seeds, such as those from Arabidopsis can be mutagenized and grown in the presence of the ABA analogs, under conditions where plant cells unable to catabolize the ABA analogs are unable to grow. Plant or plant cells where the members of the CYP72A family, or the genes encoding 7′- and 8′-ABA hydroxylase activity are altered and are now able to catabolize the ABA analogs are selected, and using the sequences provides in this specification, the modified genes can be isolated and the nature of the modification determined.

Thus, it is possible to contemplate not only the alteration of the natural catabolism of ABA using these newly described functions for the CYP72A P450 monooxygenase sequences and the exemplified 7′ and 8′-hydroxylase gene sequences, but it is within the scope of the present invention that these new 7′- and 8′-ABA hydroxylase activities, as well as the activities of related CYP72A sequences can be used to modify the catabolism of ABA analogs.

The following examples serve to illustrate the present invention but should not be regarded as limiting the scope of the invention in any way.

### EXAMPLE 1

### Cloning of the Genes for ABA 7′- and 8′-Hydroxylase

In this example, differential screening of induced and non-induced plant cells was conducted to isolate a cDNA clone encoding a ABA hydroxylase gene. The biochemical enzyme assay data (Krochko J. E., et al., 1998, Plant Physiology 118:849-860) demonstrated that there is no ABA 8′-hydroxylase activity in non-ABA-treated corn suspension cells while the maximum enzyme activity in ABA-treated corn suspension cells occurs around 16 hours after the addition of (+)-ABA. If the mRNA for ABA 8′-hydroxylase is uniquely expressed in response to ABA in this suspension cell system then there is a very strong likelihood that the gene can be isolated using a subtractive hybridization protocol. This subtractive protocol was employed to isolate an ABA hydroxylase cDNA. Black Mexican Sweet corn suspension cells were treated with 200 μM (+)-ABA for 9 hours. Total RNA was extracted from both ABA-treated and non-ABA-treated cells using a modified LiCl technique (Ausubel F. M., et al., 1994, Current Protocols in Molecular Biology, John Wiley & Sons Inc.). Poly A+ mRNA was extracted from total RNA using a Qiagen Oligotex mRNA kit (www.quiagen.com). cDNA was prepared from 2 μg of mRNA according to the directions in Clontech's PCR-Select cDNA Subtraction Kit (www.Clontech.com).

The resulting cDNA samples were subtracted according to the PCR-Select cDNA Subtraction Kit instructions resulting in 4 cDNA fractions:

1) NI/S (non-ABA-induced subtracted)

2) NI/N (non-ABA-induced non-subtracted)

3) IND/S (ABA-induced subtracted)

4) IND/N (ABA-induced non-subtracted)

After PCR amplification a portion of the subtracted ABA-induced cDNA sample (#3, IND/S) was ligated into Clontech's TA cloning vector and transformed into TOP10 F′ competent cells. cDNA inserts from individual clones of this subtracted library were PCR amplified and arrayed on nylon membranes according to the methods outlined in Clontech's PCR-Select Differential Screening kit. 420 clones were arrayed on duplicate nylon membranes and these were differentially screened using ³²P-labelled subtracted probes (#3, IND/S and #1, NI/S). cDNAs showing greater expression with the IND/S probe compared to the NI/S probe were candidate clones possibly up-regulated by (+)-ABA in corn suspension cells. These putatively ABA-regulated clones were sequenced and identities determined by BLAST searches of GenBank. In addition, cDNA clones were randomly sequenced, and later the entire subtracted library was sequenced.

### EXAMPLE 2

### Evaluation of the Cloning Procedure

The four cDNA samples (NI/S; non-ABA-induced and subtracted, NI/N; non-ABA-induced and non-subtracted, IND/S; ABA-induced and subtracted, IND/N; ABA-induced and non-subtracted) resulting from the subtractive hybridization were amplified by PCR and equivalent amounts were loaded onto an agarose gel. After transfer to nylon membrane the blot was probed successively with radiolabeled rice actin cDNA and subtracted ABA-induced cDNA (IND/S).

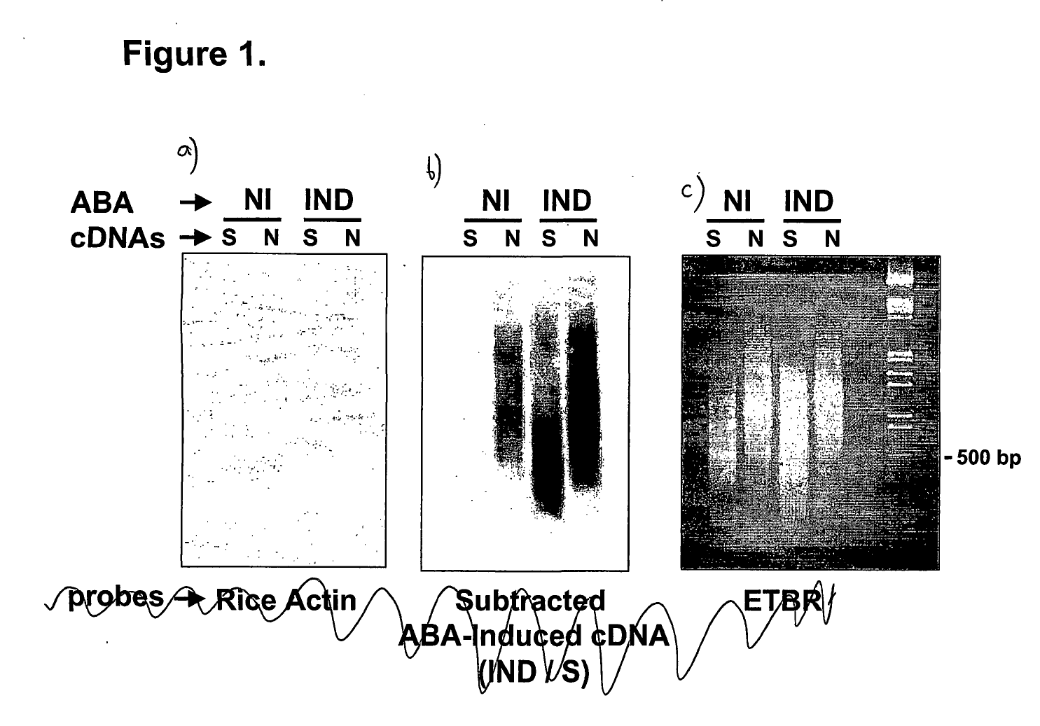

The cDNA subtraction was successful in identifying uniquely expressed genes in response to ABA induction. Actin, an abundant constitutively expressed gene was effectively removed and the cDNAs in the ABA-induced subtracted probe were very different from those in the non-ABA-treated subtracted cDNA probe. As well, the ABA-induced subtracted cDNA pool contained genes that were expressed in greater abundance in the ABA-induced cDNA pool as compared to the non-ABA-treated cDNA pool. This is shown in FIG. 1.

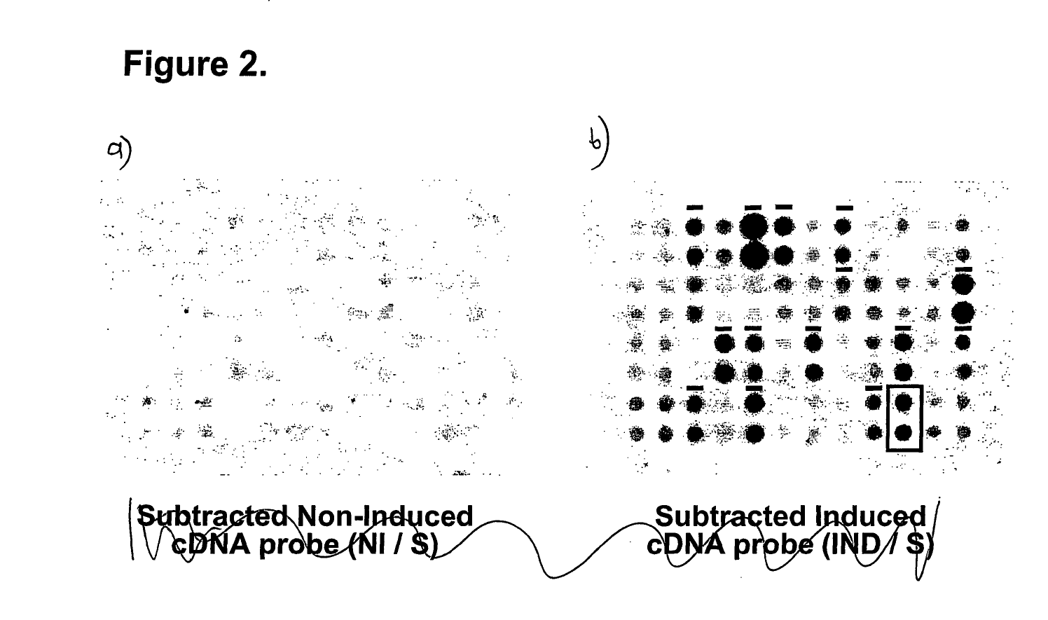

Subtracted ABA-induced cDNAs were PCR amplified and cloned into the Clontech pT-Adv vector. Clones were selected randomly from this ABA-induced subtracted library and the inserts amplified by PCR. The PCR-amplified inserts were arrayed in duplicate on nylon membranes and probed with radiolabeled subtracted non-ABA-induced cDNA (NI/S) and subtracted ABA-induced cDNA (IND/S). Clones that showed at least a 3 to 5-fold greater intensity of labeling with the IND/S cDNA probe were deemed to be upregulated by ABA in this first screen. Plasmids were sequenced. One of these, pJK6-29, was a C-terminal fragment of a cytochrome P450 monooxygenase. This clone is indicated with a box on the nylon cDNA array shown in FIG. 2.

The proportion of ABA-induced cDNAs in the subtracted library was estimated at 30-40% based on the initial differential screening and secondary screens that were carried out by spotting the individual cDNA clones on nylon membranes and screening with subtracted non-induced and induced probes as evident from FIG. 2. From the sequencing data the cDNAs in the subtracted library are mostly regulatory genes or cDNAs representing rarer mRNAs. There were no constitutively expressed abundant sequences. The upregulated genes in the ABA-induced subtracted library include genes known to be induced by ABA, for example LEAs (late-embryogenesis abundant proteins).

### EXAMPLE 3

### Isolation of a P450 Clone for an ABA-Hydroxylase

Only one P450 related sequence was isolated from the differential screening of the ABA-induced subtracted library. The sequence isolated was a 333 bp C-terminal piece including the signature P450 heme-binding region. This sequence JK6-29 is shown in SEQ ID NO.1.

### EXAMPLE 4

### Analysis of JK6-29 Expression

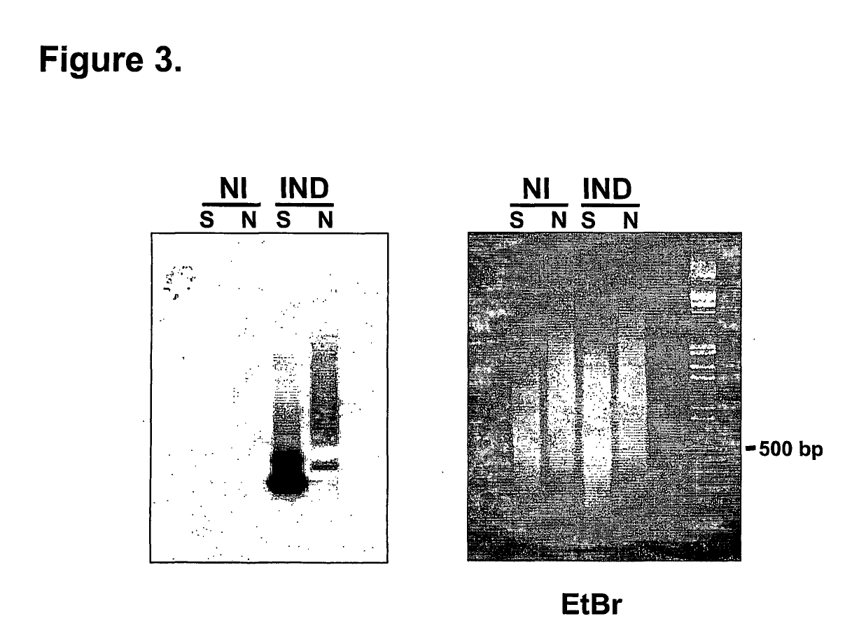

The isolated P450 cDNA (pJK6-29) was used as a probe against the four cDNA pools resulting from the subtractive hybridization (as described in example 1). This cytochrome P450 was upregulated by ABA (compare NI/N and IND/N) and successfully amplified within the subtracted ABA-induced cDNA pool (compare NI/S and IND/S). The results of this analysis are shown in FIG. 3.

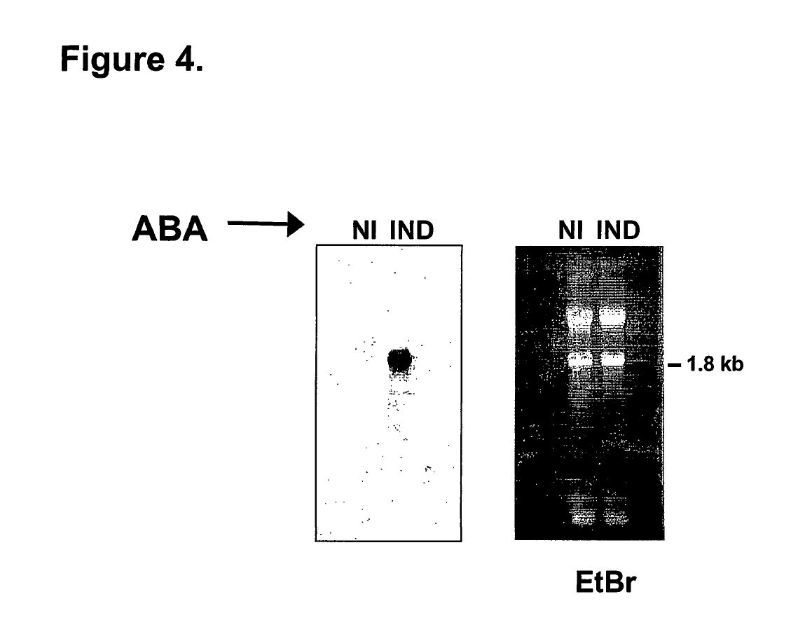

Total RNA isolated from non-ABA-induced and ABA-induced (9 hr at 2001M (+)-ABA) BMS corn suspension was probed with radiolabeled pJK6-29. The Northern blot confirmed that this P450 was upregulated by (+)-ABA in corn suspension cells (FIG. 4).

### EXAMPLE 5

### Evaluation of Expression Pattern of JK6-29 Following ABA Induction

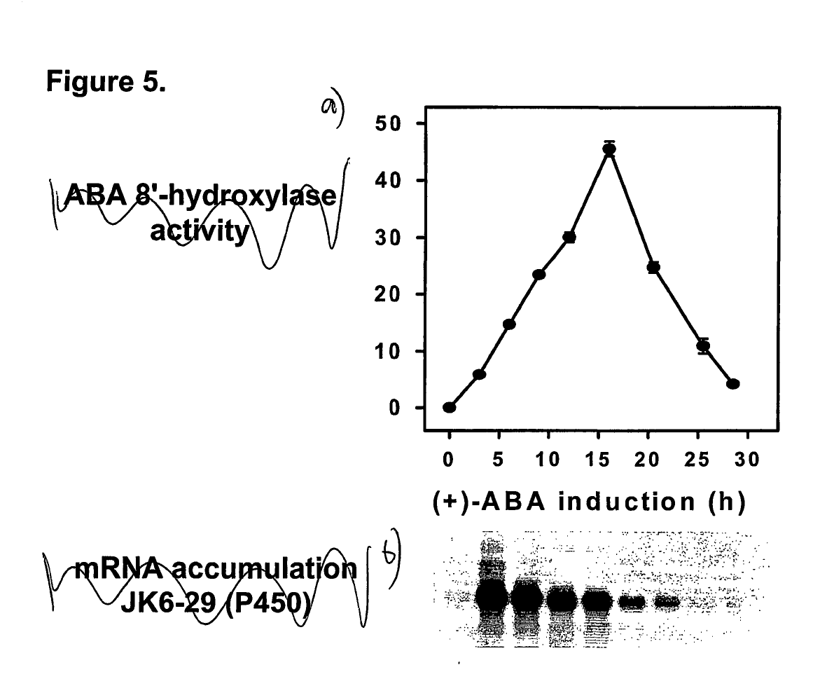

Further Northern blot analysis was carried out. In this experiment, the time course of gene expression was compared with the appearance of ABA 8′-hydroxylase activity. ABA 8′-hydroxylase activity was determined by in vitro assay (see Krochko J. E., et al., 1998, Plant Physiol 118: 849-860) with isolated microsomes isolated from BMS corn suspension cells at defined periods after the addition of 200 μM (+)-ABA. Enzyme activity was assayed at 0 (control), 3, 6, 9, 12, 16, 20.5, 25.5 and 28.5 hours after the addition of (+)-ABA. Total RNA was extracted from duplicate tissue samples and expression of the P450 clone, pJK6-29, was determined by Northern blot analysis. The time-course of mRNA induction for pJK6-29 is consistent with the appearance of ABA 8′-hydroxylase activity (FIG. 5).

The pattern of accumulation of the mRNA corresponding to this cDNA in corn suspension cells after the addition of (+)-ABA confirmed that this P450 enzyme was transcriptionally regulated by (+)-ABA and that the expression of its mRNA was short-lived (FIG. 5). The mRNA induction profile was consistent with the enzyme activity profile. Messenger RNA accumulation was strongest soon after the addition of ABA (3 hours). At every time point thereafter the expression of this P450 mRNA was progressively diminished. The data suggest that gene transcription is induced as a pulse of activity soon after the addition of (+)-ABA (200 μM). Messenger RNA accumulation declined rapidly after this initial burst of synthesis and following the decline in mRNA levels the enzymatic activity demonstrated a similar decline.

### EXAMPLE 6

### Isolation of Full-Length Copies of JK6-29

A full-length sequence for the corn CYP72A cDNA has been obtained by a sib-selection strategy (Alfandari D. and Darribere T., 1994, In PCR Methods and Applications, pp 46-49, Cold Spring Harbour) from a full-length cDNA library made from RNA extracted from ABA-induced BMS corn suspension cells, screened by PCR with primers designed specifically for the JK6-29 sequence.

These primers are:

| | |
|---|---|
| SEQ ID NO:7: | 5′ CGTTGGAGGATGGTGCATAG 3′ and |
| SEQ ID NO:8: | 5′ GCTCGGTGGAATCAAATATCC 3′. |

The putative full-length sequence was cut out of pYES2 (pBE10-30, EcoRI/NotI insert) with BamHI and NotI and subcloned into pT-Adv (Clontech) (pBE10-30-3) and was sequenced first using vector primers to determine whether it was full-length. On the basis of this sequence information and sequences of partial length clones the following sequencing primers were designed.

| | |
|---|---|
| SEQ ID NO:9: | 5′ GATTCCTGACCCAGAGTTAG 3′, |
| SEQ ID NO:10 | 5′ GCAAGGAGAACTAGCTGAAC 3′, |
| SEQ ID NO11: | 5′ CCGGATTATGATAGCTTGAG 3′, |
| SEQ ID NO:12 | 5′ CCTGTGCTTTGCCCATT 3′, |
| SEQ ID NO:13 | 5′ CGCAACCAGGCATCAG 3′. |

The full-length cDNA sequence is given in SEQ ID NO:2.

Comparison of this full-length sequence pBE10-30-3 confirmed that this P450 contains the pJK6-29 sequence and is related to the CYP72A gene family. cDNA was PCR-amplified from plasmid pBE10-30-3, adding BamHI and EcoRV sites to the sequence at the N-terminal and C-terminal ends, respectively. Primers used are:

| | |
|---|---|
| Forward primer, | |
| 5′ATGGATCCATGCTGCGGGAAGTCTCTC 3′, | SEQ ID NO:14 |
| Reverse primer | |
| 5′ ACGATATCGTCCGGTAGCAGCATAGAAG 3′). | SEQ ID NO:15 |

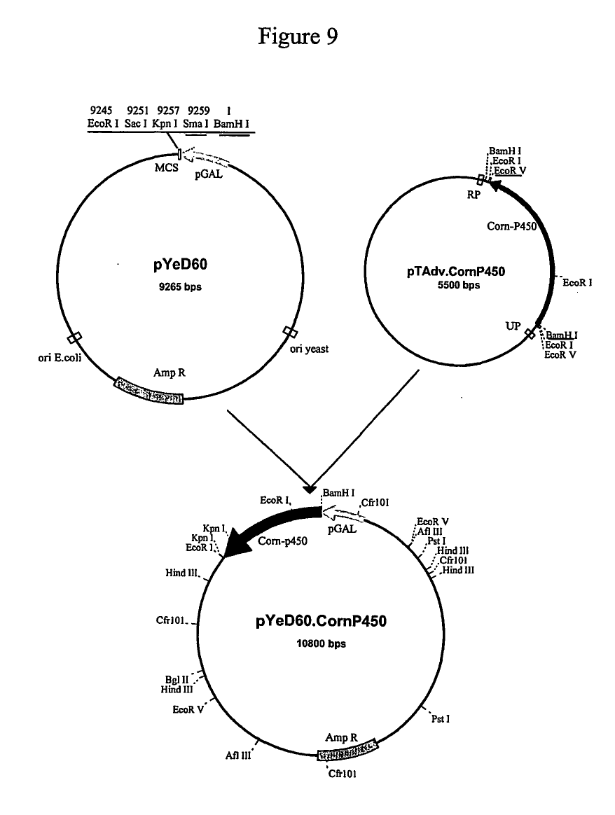

PCR conditions were 94° C. for 30 sec, then 5 cycles of 94° C./30 s, 50° C./30 s, 68° C./120 s and 25 cycles of 94° C./30 s, 60° C./30 s, 68° C./120 s followed by an extension step at 68° C. for 3 minutes, using Clontech's Advantage 2 Polymerase Mix. The PCR product was TA cloned into pT-Adv (Clontech) and the sequence confirmed by sequencing (pTAdv.CornP450). Vector pYeDP60 was cut with BamHI and SmaI and the cornP450 insert was cut from pTAdv.CornP450 with BamHI and EcoRV and gel-purified. The vector and insert were ligated to make YeDP60.CornP450 (FIG. 9) and the orientation and sequence fidelity were confirmed by sequencing again.

### EXAMPLE 7

### Isolation of Homologous Clones from Other Plant Species

Using the corn JK6-29 cDNA sequence as the standard, orthologous sequences from Arabidopsis were identified by screening the Arabidopsis database. The related genes in Arabidopsis belong to a large (9 members) cytochrome P450 gene subfamily whose members are arrayed in tandem on chromosome 3. The sequence information on this series of P450 monooxygenase genes was first submitted to GenBank by the Kazusa DNA Research Institute on Feb. 1, 1999 (P1 clone MIE1; GenBank AB023038). The original submission did not describe a function for these P450 gene sequences.

Several P450 ESTs belonging to the CYP72A P450 subfamily were obtained from ABRC (Arabidopsis Biological Resource Center) and the inserts were sequenced and the sizes determined. These P450s included full-length sequences for CYP72A7 and CYP72A14. The CYP72A7 (Ath30): clone 187N22T7 received from ABRC (GB R90024) was analyzed as follows. Full-length of the coding sequence was confirmed initially by sequencing using vector primers, followed by sequencing with gene-specific primers.

cDNA was PCR-amplified from the ABRC plasmid 187N22T7 with gene-specific PCR primers, adding BamHI and KpnI sites to the N-terminal and C-terminal ends, respectively using the following primers.

| | |
|---|---|
| A7-Forward-2 primer | |
| 5′ ATCAGGATCCGTGAGAATCGGAGATGTC 3′ | SEQ ID NO:16 |
| and A7-Reverse-2 primer | |
| 5′ TCGTGGTACCAAATCAGAGCTTGTGCAG 3′ | SEQ ID NO:17 |

PCR conditions were 94° C. for 30 sec, then 5 cycles of 95° C./30 s, 50° C./30 s, 68° C./150 and 20 cycles of 95° C./30 s, 68° C./180 s, followed by an extension step at 68° C. for 5 minutes, with Advantage 2 Polymerase Mix (Clontech). The pYEDP60 vector and PCR product were cut separately with BamHI and KpnI, and then the linearized vector and PCR product were gel-purified.

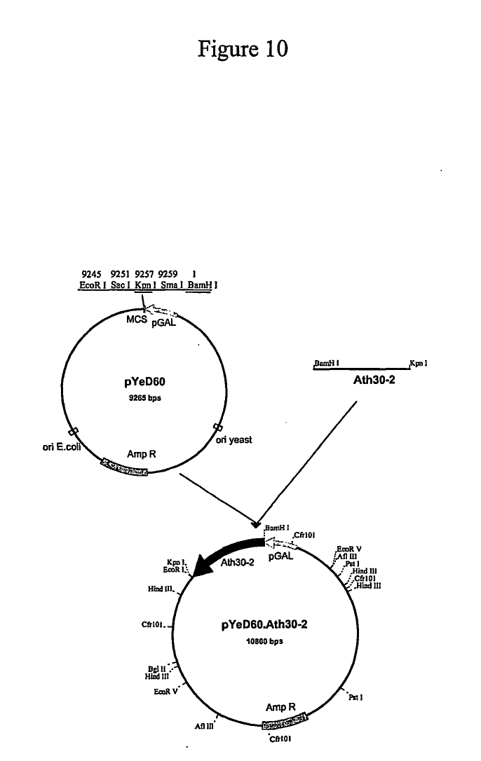

The PCR product was ligated into the cut vector and transformed into TOP10F′ competent cells. Plasmid pYeDP60.Ath30-2 (FIG. 10) was isolated from selected colonies and the insert confirmed by sequencing.

The CYP72A14 (Ath29): clone 181D14T7 received from ABRC (GB H36956) was similarly analyzed. Full-length was confirmed initially by sequencing using vector primers, followed by sequencing with gene-specific primers.

cDNA was PCR-amplified from the ABRC plasmid 181D14T7 using gene-specific PCR primers adding KpnI and EcoRI sites to the sequence at the N-terminal and C-terminal ends, respectively. Primers used:

| | |
|---|---|
| A14-Forward-1 primer | |
| 5′ GTGGTACCATGGAGATATCAGTTTCTTCG 3′ | SEQ ID NO:18 |
| and A14-Reverse-1 primer | |
| 5′CTGAATTCATGTGATTAGAGCTTGTGCAG 3′. | SEQ ID NO:19 |

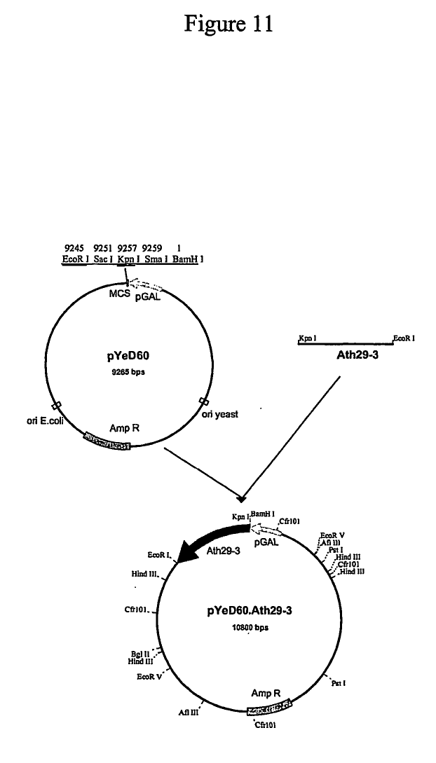

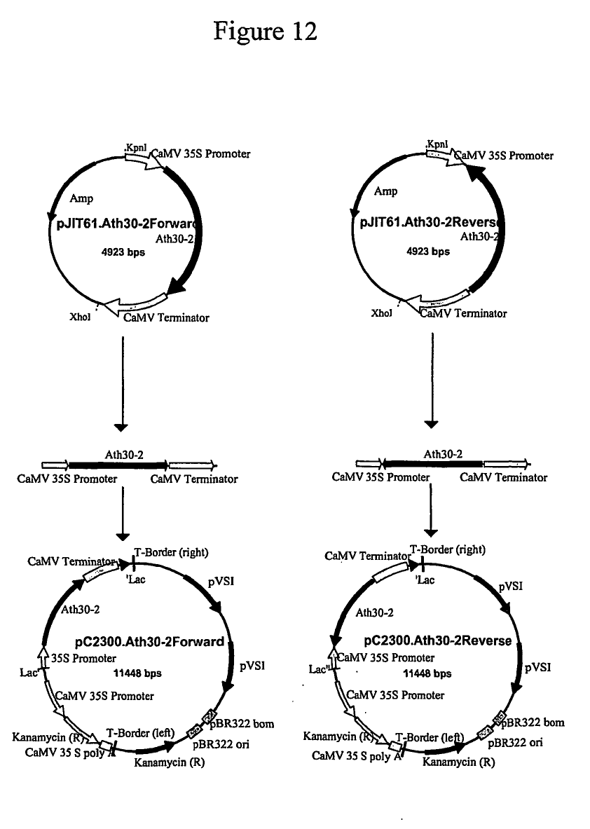

PCR conditions were 94° C. for 30 sec, then 5 cycles of 95° C./30 s, 55° C./30 s, 68° C./150 s and 20 cycles of 95° C./30 s, 68° C./180 s, followed by an extension step at 68° C. for 5 minutes, using Clontech's Advantage 2 Polymerase Mix. The pYEDP60 vector and PCR product were cut separately with KpnI and EcoRI, and then the PCR products gel-purified. The PCR product was ligated into the cut vector and transformed into TOP10F′ competent cells. Plasmid pYeDP60.Ath29-3 (FIG. 11) was isolated from selected colonies and the insert confirmed by sequencing.

### EXAMPLE 8

### Expression of ABA Hydroxylase Genes in Heterologous Systems

For yeast expression, the cDNAs were transferred into the expression vector pYeDP60 (Pompon D., et al., 1996, Method Enzymol 272: 51-64), and purified recombinant plasmid was subsequently transformed into WAT 11 and WAT 21 yeast strains over-expressing the Arabidopsis cytochrome P450 reductases, CTR1 and CTR2, respectively (Pompon et al., ibid).

Plasmid was isolated from pYeDP60.CornP450 (FIG. 9), pYeDP60.Ath30-2 (FIG. 10) and pYeDP60.Ath29-3 (FIG. 11) and used to transform yeast strains, WAT 11 and WAT21, using a Li Acetate technique (Kohlami S. E., et al., 1997, In Differentially Expressed Genes in Plants, Taylor and Francis, pp 63-83). Transformants were selected on SC minus URA (6.7 g/L yeast nitrogen base without amino acids (BIO-101), 1.8 g/L uracil drop-out powder) containing 2% Glucose. Yeast transformation was confirmed by PCR.

For expression analysis the cultures were grown for two days on SC minus URA agar plates at 30° C. A colony was then transferred to liquid culture in YPG (10 g/L yeast extract, peptone 10 g/L, glucose 20 g/L) for growth overnight at 30° C. with shaking. The following day the culture was collected by centrifugation (5000 rpm for 10 minutes), washed twice with YPL (yeast extract 10 g/L, peptone 10 g/L, galactose 20 g/L), and diluted to OD 0.5 in YPL (Bishop G. J., et al., 1999, Proc Natl Acad Sci 96: 1761-1766). (+)-ABA was added at 20 μM to 50 μM. The cultures were incubated for 24 hours at 30° C. with shaking. At the conclusion of the experiment the culture media was collected by centrifugation of the entire culture volume at 5000 rpm for 15 minutes. The media was acidified with HCL (0.166 N final concentration) and re-centrifuged to remove additional protein contamination. One to 10 mls of the sample was loaded onto an Oasis HLB column (Waters) to purify ABA and ABA-metabolites and the resulting eluant was analyzed by LC/MS/MS.

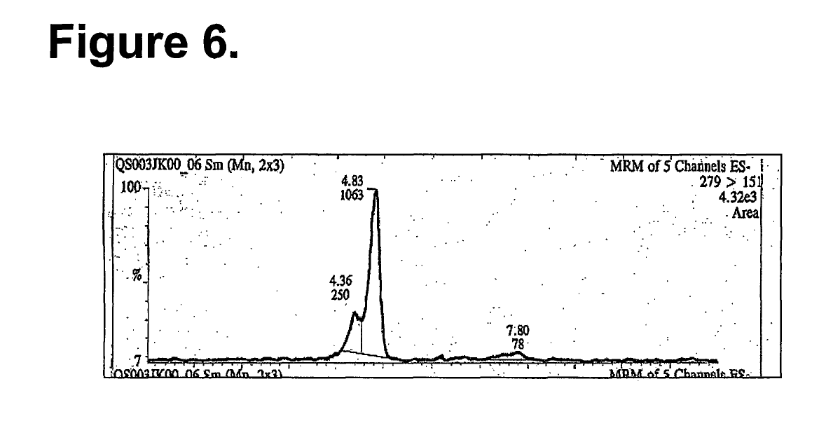

ABA 7′-hydroxylase activity was demonstrated by LC/MS/MS in the cultures in which the CYP72A7 gene was inserted in the pYeDP60 vector (pYeDP60.Ath30-2) and this construct transformed into the WAT11 yeast strain (FIG. 6). The WAT21 yeast strain did not support any ABA hydroxylase activity in these experiments.

### EXAMPLE 9

### Construction of a Plant Transformation Vectors Using the CYP72A7 Gene

For constitutive over-expression and anti-sense expression in plants the cDNAs were cloned into a 35S expression cassette in pJIT61 (received from Roger Hellens and Phil Mullineaux; http://www.pgreen.ac.uk/). The expression cassette containing the gene of interest was then subcloned into the MCS of the binary vector pCAMBIA 2300 (received from CAMBIA; http://www.cambia.org/). The insert was confirmed by restriction digests and the resultant plasmid construct was used to transform Agrobacterium strain MP90.

To make the 35S.CYP72A7 expression constructs for Arabidopsis/plant transformation, the yeast expression construct pYeDP60.Ath30-2 was cut first with KpnI and BamHI. The Ath30-2 insert was gel-purified and blunt-ended with T4 DNA polymerase. pJIT61 vector was cut with SmaI to give a linearized and blunt-ended vector and was dephosphorylated using CIAP (calf intestinal alkaline phosphatase). The Ath30-2 fragment was ligated into the pJIT61 vector and transformed into E. coli TOP10F′. Purified plasmid was checked for orientation and both Ath30-2Forward and Ath30-2Reverse colonies were chosen (pJIT61.Ath30-2Forward, pJIT61.Ath30-2Reverse) (FIG. 12). The pJIT61.Ath30-2Forward and pJIT61.Ath30-2Reverse plasmids were cut with XhoI, blunt-ended with T4 DNA polymerase and re-cut with KpnI to liberate the 35S.Ath30-2Forward and 35S.Ath30-2Reverse cassettes. These fragments were gel-purified. pCAMBIA2300 was cut with HindIII, blunt-ended and re-cut with KpnI. The 35S-P450 cassettes were ligated into pCAMBIA2300 to give plasmids pCAMBIA2300.Ath30-2Forward and pCAMBIA2300.Ath30-2Reverse (FIG. 12). These plasmids were transformed into Agrobacterium strain MP90 and these cultures were used to transform Arabidopsis thaliana (strain Columbia) by the floral dip method (Clough S. J and Bent A. F., 1998, Plant J 16: 735-743). Plants were obtained and analyzed for the presence of the inserted gene.

### EXAMPLE 10

### Construction of a Plant Transformation Vectors Using the CYP72A14 Gene

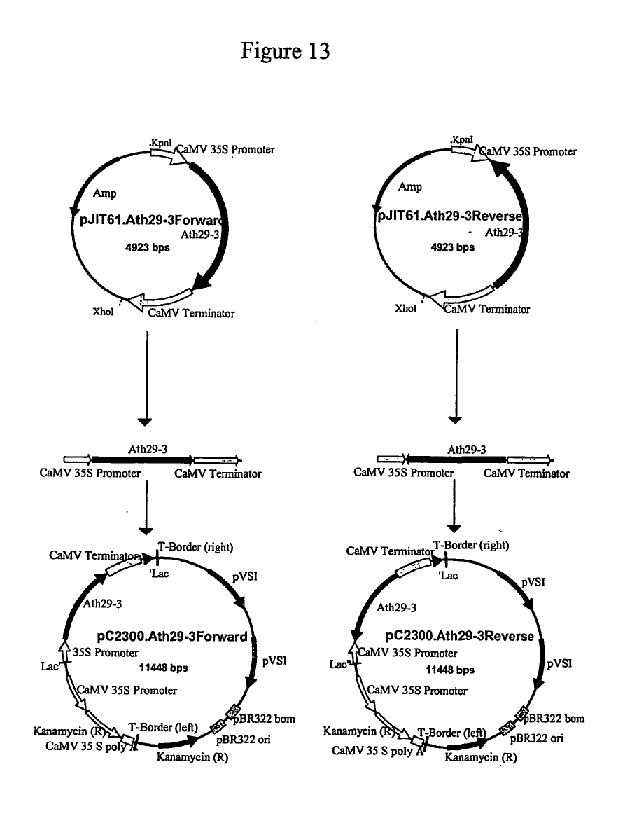

To make the 35S.CYP72A14 expression constructs for Arabidopsis/plant transformation, the yeast expression construct pYeDP60.Ath29-3 was cut with KpnI and EcoRI, and the insert was gel-purified and blunt-ended with T4 DNA polymerase. pJIT61 was cut with SmaI to give a linearized and blunt-ended vector and was dephosphorylated using CIAP (calf intestinal alkaline phosphatase). The Ath29-3 fragment was ligated into the pJIT61 vector and transformed into E. coli TOP10F′. Purified plasmid was checked for orientation and both Ath29-3Forward and Ath29-3Reverse colonies were chosen (pJIT61.Ath29-3Forward, pJIT61.Ath29-3Reverse) (FIG. 13). The pJIT61.Ath29-3Forward and pJIT61.Ath29-3Reverse plasmids were cut with XhoI, blunt-ended with T4 DNA polymerase and re-cut with KpnI to liberate the 35S.Ath29-3Forward and 35S.Ath29-3Reverse cassettes. These fragments were gel-purified. pCAMBIA2300 was cut with HindIII, blunt-ended and re-cut with KpnI. The 35S-P450 cassettes were ligated into pCAMBIA2300 to give plasmids pCAMBIA2300.Ath29-3Forward and pCAMBIA2300.Ath29-3Reverse (FIG. 13). These plasmids were transformed into Agrobacterium strain MP90 and these cultures were used to transform Arabidopsis thaliana (strain Columbia) as described above. Plants were obtained and analyzed for the presence of the inserted gene.

### EXAMPLE 11

### Construction of a Plant Transformation Vectors Using the corn CYP72A Gene

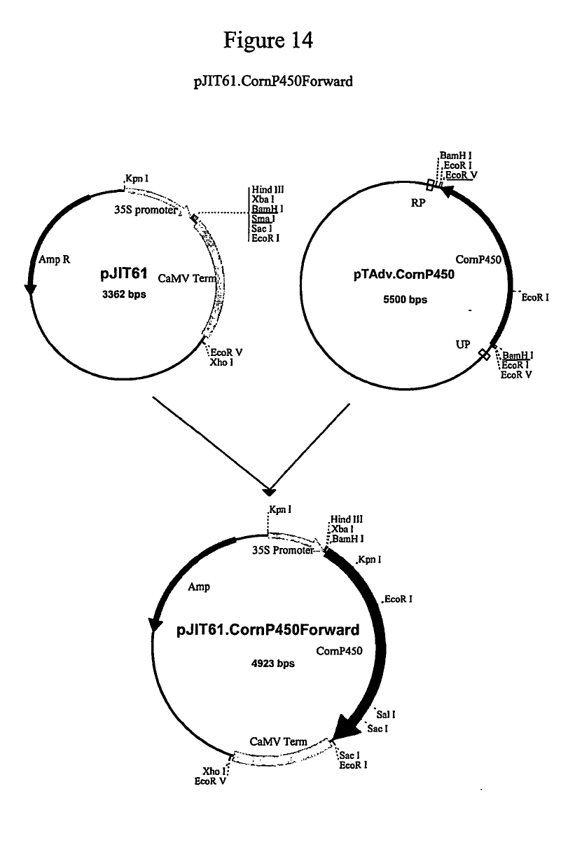

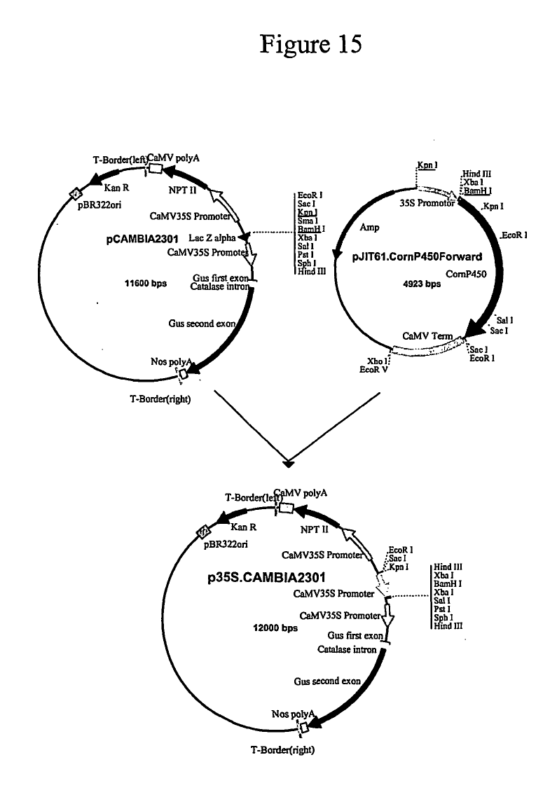

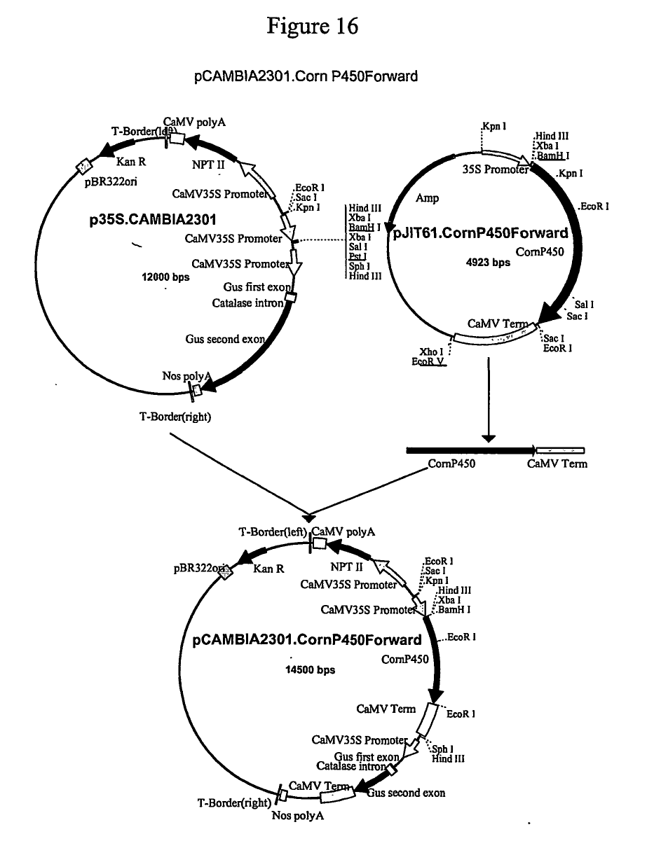

To make the 35S.CornP450Forward expression constructs for Arabidopsis/plant transformation, the pTAdv.CornP450 was cut with EcoRV and BamHI, and the insert was gel-purified. pJIT61 was cut with SmaI and BamHI. The CornP450 fragment was ligated into the linearized pJIT61 vector and transformed into E. coli TOP10F′ to give pJIT61.CornP450Forward (FIG. 14). The 35S promoter was removed from pJIT61.CornP450Forward by KpnI and BamHI digestion, gel-purified and ligated to pCAMBIA2301 previously cut with KpnI and BamHI to give p35S.CAMBIA2301 (FIG. 15). After transformation, colony selection and plasmid preparation the p35S.CAMBIA2301 was cut with PstI, blunt-ended with T4 DNA polymerase and re-cut with BamHI. The CornP450Forward.CaMVTerm was isolated from pJIT61.CornP450Forward by digestion with BamHI and EcoRV and gel-purified. The CornP450Forward.CaMVTerm piece was ligated into the linearized p35S.CAMBIA2301 to reconstitute the 35S.P450CornForward expression cassette in the binary vector pCAMBIA2301. Purified plasmid (pCAMBIA2301.CornP450Forward) (FIG. 16) was prepared, sequenced and transformed into Agrobacterium strain MP90.

To make the 35S.CornP450Reverse expression construct the cDNA was PCR amplified from plasmid BE10-30-3 using gene-specific primers that added HpaI and XbaI sites to the N-terminal and C-terminal, respectively:

| | |
|---|---|
| cornPCR-N: | |
| 5′ACGTTAACATGCTGCGGGAAGTCTCTC 3′ | SEQ ID NO:20 |
| cornPCR-C: | |
| 5′ACTCTAGACCGGTAGCAGCATAGAAGGA 3′ | SEQ ID NO:21 |

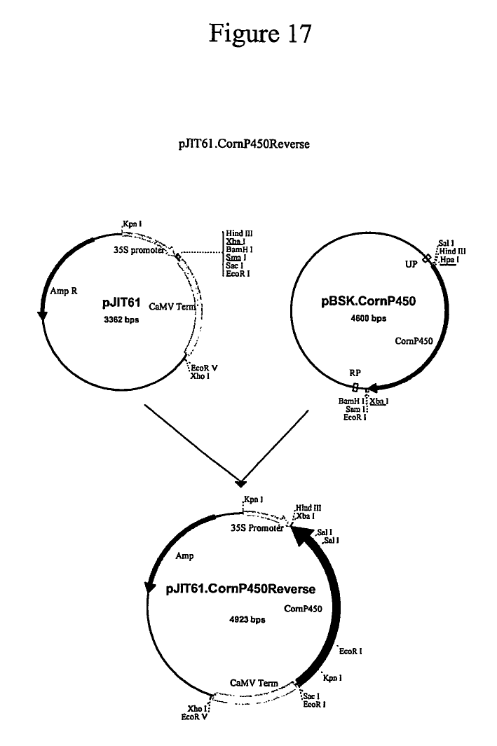

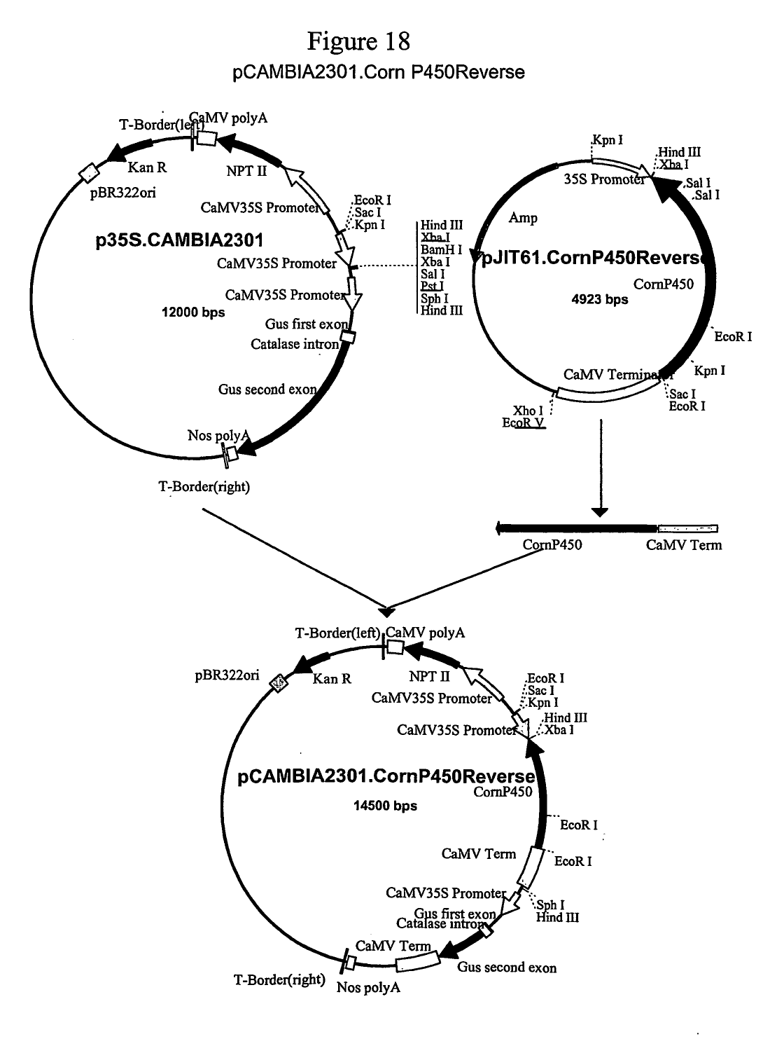

PCR conditions were 94° C. for 3 minutes, 5 cycles of 94° C./30s, 48° C./30 s, 72° C./120 s, 25 cycles of 94° C./30 s, 57° C./30 s, 72° C./120 s, and 72° C. for 3 minutes using PfuTurbo (Stratagene). The product was ligated into EcoRV cut dephosphorylated pBluescript SK(+), and transformed into TOP10F′ to give pBSK.CornP450 (FIG. 17). Colonies were selected and sequenced using gene-specific primers, as before, to ensure fidelity of the PCR amplification. The cornP450 insert was excised from pBSK.CornP450 by cutting with HpaI and XbaI and gel-purified. The purified insert was ligated to XbaI and SmaI cut pJIT61 vector to give pJIT6 I.CornP450Reverse (FIG. 17). This vector was cut with XbaI and EcoRV and the CornP450Reverse.CaMVTerm piece was gel-purified. The CornP450Reverse.CaMVTerm was ligated into p35S.CAMBIA2301 that had been cut with PstI, blunt-ended and then cut again with XbaI. The structure of the resultant vector pCAMBIA2301.CornP450Reverse (FIG. 18) was verified by restriction digests, sequenced and trnsformed into Agrobacterium strain MP90.

### EXAMPLE 12

### Construction of Plant Transformation Vectors Using an Inducible Promoter and a CYP72A Gene

It some instances it is desirable to have a CYP72A gene under the control of a inducible promoter. For conditional tetracycline-inducible expression in tobacco, the cDNAs were cloned into the binary vector pBINHygTX (Gatz C., et al., 1992, Plant J 2: 397-404). This promoter is repressed in plants expressing the TetR gene but de-repressed when tetracycline is applied to the plant (Gatz et al., ibid). In this way specific temporal and spatial expression can be achieved. Tobacco seeds from plants already expressing TetR were received from Christiane Gatz. Expression of TetR was confirmed by Northern blot expression analysis.

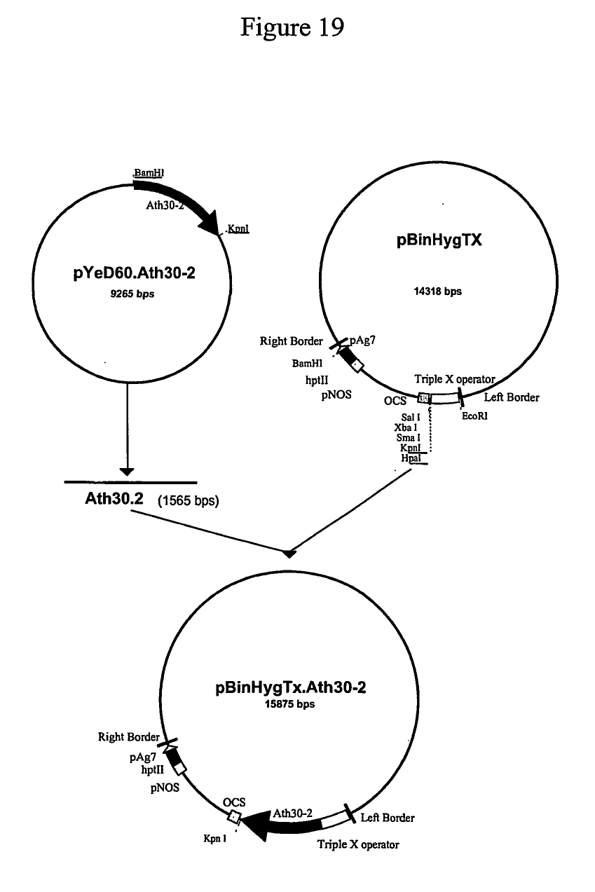

For the CYP72A7-containing construct, the vector pYeDP60.Ath30-2 was cut with BamHI, blunt-ended with T4 DNA polymerase and the Ath30-2 gene insert was liberated by a final cut with KpnI. The Ath30-2 insert was gel-purified. pBINHygTx was cut with HpaI and KpnI sequentially and the Ath30-2 insert ligated to the HpaI/KpnI cut vector to give pBINHygTx.Ath30-2 (FIG. 19). The plasmid construction was confirmed by restriction digestion and sequencing.

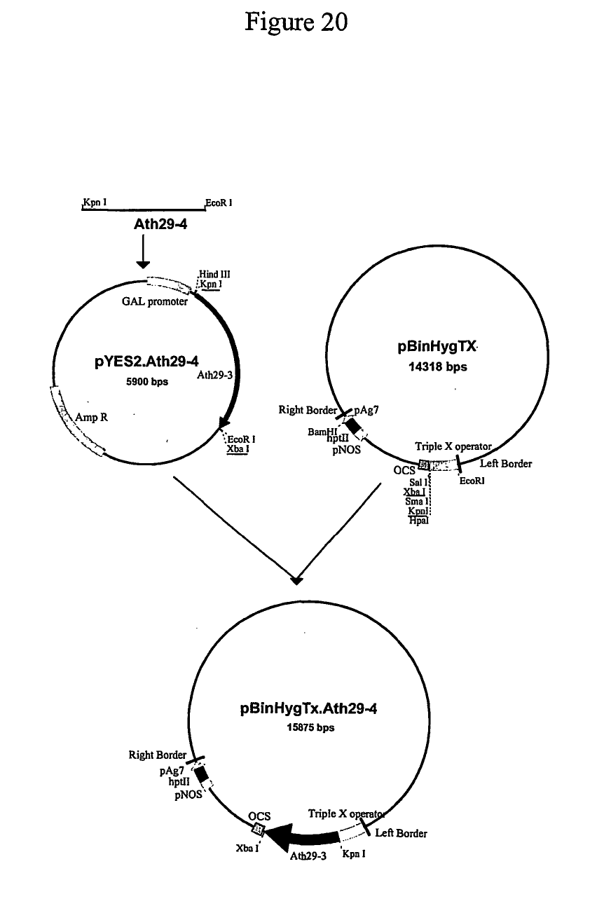

For the CYP72A14-containing construct the gene was obtained from pYES2.Ath294 (pYES2.Ath29-4: sequence was PCR amplified (as described for pYeDP60.Ath29-3), cut with EcoRI and KpnI to expose the ends, and ligated into pYES2 vector cut with EcoRI and KpnI). pYES2.Ath29-4 was cut with XbaI and KpnI and the gel-purified insert was ligated into XbaI/KpnI cut pBINHygTx. The resultant plasmid, pBINHygTx.Ath294 (FIG. 20), was confirmed by restriction digestion and sequencing.

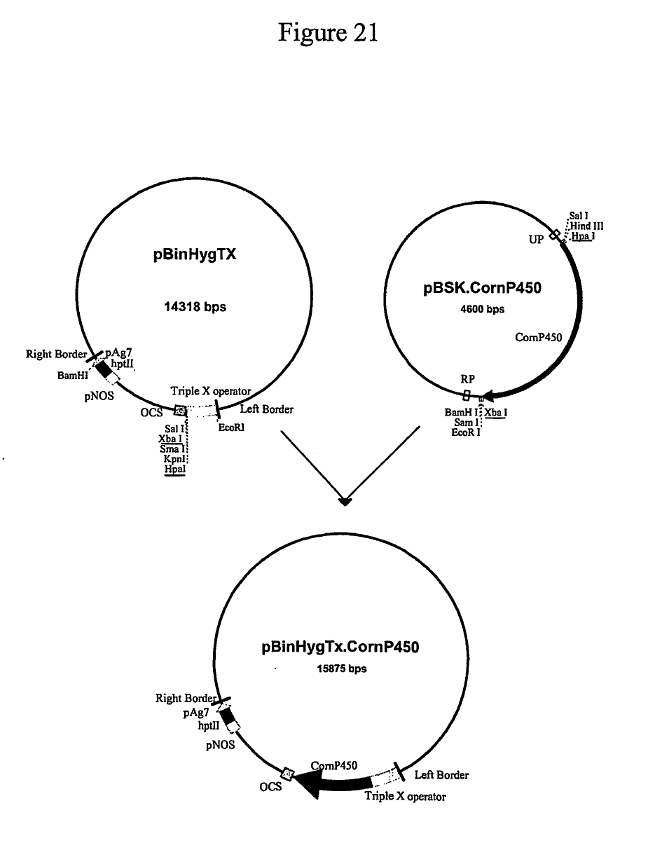

For the CornP450-containing construct the CornP450 insert was cut from pBSK.CornP450 with HpaI and XbaI, gel-purified and ligated into pBINHygTx vector cut with HpaI and XbaI to give pBINHygTx.CornP450 (FIG. 21). This plasmid was purified and the plasmid construction confirmed by restriction digestion and sequencing.

After plasmid purification, the plasmids pBINHygTx.Ath30-2, pBINHygTx.Ath29-4 and pBINHygTx.CornP450 were transformed into Agrobacterium MP90. Successful Agrobacterium transformation was confirmed by restriction digestion of isolated plasmid.

### EXAMPLE 13

### Transformation of Plant Cells with a Vector Containing an Inducible Promoter and a CYP72A Gene

The vectors pBINHygTx.Ath30-2, pBINHygTx.Ath29-4 and pBINHygTx.CornP450 were used to re-transform tobacco, already expressing the TetR. Tobacco plants were selected on hygromycin due to the presence of kanamycin resistance in the TetR plants. Plants were obtained and analyzed for the presence of the inserted gene.

Sequence Listing Free Text

| | |
|---|---|
| SEQ ID NO. 7 | JK6-29 PCR primer |
| SEQ ID NO. 8 | JK6-29 PCR primer |
| SEQ ID NO. 9 | Sequencing primer |
| SEQ ID NO. 10 | Sequencing primer |
| SEQ ID NO. 11 | Sequencing primer |
| SEQ ID NO. 12 | Sequencing primer |
| SEQ ID NO. 13 | Sequencing primer |
| SEQ ID NO. 14 | BE10-30-3 PCR primer (BamH I) |
| SEQ ID NO. 15 | BE10-30-3 PCR primer (EcoR V) |
| SEQ ID NO. 16 | CYP72A7 (Ath30) PCR primer (BamH I) |
| SEQ ID NO. 17 | CYP72A7 (Ath30) PCR primer (Kpn I) |
| SEQ ID NO. 18 | CYP72A14 (Ath29) PCR primer (Kpn I) |
| SEQ ID NO. 19 | CYP72A14 (Ath29) PCR primer (EcoR I) |
| SEQ ID NO. 20 | BE10-30-3 PCR primer (Hpa I) |
| SEQ ID NO. 21 | BE10-30-3 PCR primer (Xba I) |
| SEQ ID NO. 22 | Conserved motif |
| SEQ ID NO. 23 | Conserved motif |
| | In position 5, Xaa = Lys, Arg or His |
| | In position 6, Xaa = Any amino acid |
| SEQ ID NO. 24 | Conserved motif |
| | In position 2, Xaa = Leu or Ile |
| | In position 5, Xaa = Leu, Ile or Val |
| | In position 6, Xaa = Leu or Met |
| | In position 7, Xaa = Val or Leu |
| | In position 8, Xaa = Glu, Gln or lys |

Nucleotide and Peptide Sequences

| | |
|---|---|
| pJK6-29 | |
| cDNA | |
| ACGGCGCGTGGGTGTAGGATGGCGAGAGCTCGAACGAGAAGCGTTGGAGGATGGTGCATAGCGT | SEQ ID NO:1 |
| CATCTTGGCTTCCAGCAACGCAAAGCTCTGGCCGATGCAGATTCTAGGCCCCCCTCCGAACGGA | |
| AAGAAAGCAGCCTGATGCCTGGTTGCGCTGGAGATGCCGTTGGCAAACCTTTCTGGGTTGAACT | |
| CGCTTGCGTCTTTTCCCCAAATGTCAGGATCATGGTGAATGAAGATGACGGGTAGAAGGACGTC | |
| AACTCCTGCAGGATATTTGATTCCACCGAGCTCCATTTCCTTATAAGTTCTTCTTGTTAGAAAG | |
| GTTGCCGGTGGGT | |
| pBE10-30-3 | |
| cDNA | |
| GGCTGTATCCGCCATCTGCTTCTCTCTTCCACTGCCCCAAGCCGCCACCAATGGCGACCTGCGT | SEQ ID NO:2 |
| TCTGCTGATGCTGCGGGAAGTCTCTCCGTGGGCGCTGGCCAGCGTGGTGGCGTCCGTGTCGCTG | |
| TTGTGGCTGGTGGTCTGGACGCTGGAGTGGGCCTGGTGGACGCCTTGGCGGCTCGAGCGGGCCC | |
| TGCGGGTCCAGGGCCTCAAGGGCACCAGGTACCGCCTCTTCACCGGCGACCTGAGGGAAACCGC | |
| CCGGGCTAACCGGGAGGCTCGCAAGAAGCCGCTGCCGCTCGGCAGCCACGACATCGCCCCACGC | |
| GTGCAGCCCATGCATCACAGCACCATCAAGGAATACGGGAAACTATCGTTCACCTGGTTCGGCC | |
| CAACACCAAGGGTGATGATTCCTGACCCAGAGTTAGTCAAAGAGGTGCTGTCTAATAAGTTTGG | |
| CCACTTTGGCAAACCAAGGAGTAACCGCATTGGGAGGTTGCTAGCCAACGGGCTTGTAAATCAT | |
| GATGGTGAAAAATGGGCAAAGCACAGGAGAATTCTTAATCCTGCATTTCACCATGAAAAAATAA | |
| AGGGGATGATGCCAGTATTTTCTACCTGCTGTATTGAAATGATTACTAGATGGGATAATTCAAT | |
| GCCTTCTGAGGGATCTTCTGAGATAGATGTCTGGCCTGAGTTCCAGAATCTTACTGGAGATGTT | |
| ATCTCAAGAACTGCGTTTGGGAGCAACTATCAAGAAGGGAGGAGAATTTTCGAGCTGCAAGGAG | |
| AACTAGCTGAACGCCTCATCCAATCTGTTCAGACAATATTTATCCCAGGCTATTGGTTCTTGCC | |
| CACCAAAAACAACAGAAGGATGAGAGCGATCGATGTAGAGATCCGCAAAATTCTCCGTGAAATA | |
| ATTGGGAAGAGAGACAAGGATACTAAAAACAGAGAAACAAATAACGACGACTTGCTGGGCTTAT | |
| TACTGGAGTCAAACACAAGGCAATCAAATGGAAATGCAAGCCTGGGATTGACAACAGAAGATGT | |
| GATTGAGGAATGCAAGTTATTTTACTTTGCAGGTATGGAGACAACATCAGTCCTTCTTACTTGG | |
| ACACTTATTGTGCTAAGCATGCACCCAGAATGGCAAGAGAGAGCAAGAGAAGAGGTTTTGAGCC | |
| ACTTTGGAAGAACCACACCGGATTATGATAGCTTGAGCCGCCTCAAGACTATAACCATGATTCT | |
| ACATGAGGTCCTTAGGTTGTACCCACCGGCAACCTTTCTAACAAGAAGAACTTATAAGGAAATG | |
| GAGCTCGGTGGAATCAAATATCCTGCAGGAGTTGACCTCCTTCTACCCGTCATCTTCATTCACC | |
| ATGATCCTGACATTTGGGGAAAAGACGCAAGCGAGTTCAACCCAGAAAGGTTTGCCAACGGCAT | |
| CTCCAGCGCAACCAGGCATCAGGCTGCTTTCTTTCCGTTCGGAGGGGGGCCTAGAATCTGCATC | |
| GGCCAGAGCTTTGCGTTGCTGGAAGCCAAGATGACGCTATGCACCATCCTCCAACGCTTCTCGT | |
| TCGAGCTCTCGCCATCCTACACCCACGCGCCGTACACCGTGATAACACTGCACCCTCAACATGG | |
| TGCTCAGATAAGGCTCAAAAAGCTTTCTCCGTGATGCTCCTTCTATGCTGCTACCGGACACTAC | |
| TTTCGTTACTGACCGCATATGTAGAAACGTATTTCTTATTTAGTATGTATTTTTTAGGATATAA | |
| ATAAAAAGATGGTGCATATTAATGGGAAATAAGTTCCCTTGTATGCATTGCGATGTAATTTTGG | |
| GAAGATTTGCAAGGAACTTAATTATACAATATATGTATTG | |
| CYP72A7: (Ath30-2) | |
| DNA | |
| ATGTCTTTTTCAGTAGTAGCAGCTTTACCGGTGCTTGTAGCAGTAGTGGTACTGTGGACATGGC | SEQ ID NO:3 |
| GGATCGTGAAGTGGGTCTGGATAAAACCAAAGATGCTTGAGAGTTCTTTGAAAAGACAGGGTCT | |
| TACCGGAACTCCTTACACTCCTCTCGTCGGAGATATAAAGAGGAATGTTGATATGATGATGGAA | |
| GCGAGATCTAAACCCATCAATGTAACGGATGATATCACCCCACGTCTCCTTCCTCTTGCCTTAA | |
| AGATGCTCAATTCTCACGGAAAGACTTTCTTCATATGGATTGGACCACTTCCAACGATTGTGAT | |
| AACGAATCCTGAGCAGATCAAGGAAGTCTTTAATAAAGTCAACGACTTTGAGAAAGCTTCTACA | |
| TTCCCTTTGATCAGATTGTTAGCAGGTGGGCTTGCAAGTTACAAGGGAGATAAATGGGCGAGTC | |
| ACAGGAGGATCATCAACCCGGCTTTTCACCTCGAAAAAATCAAGAACATGATCCCTGCGTTCTA | |
| CCATTGTTGCAGCGAGGTTGTCTGTCAATGGGAGAAGCTATTTACAGATAAAGAATCGCCTCTT | |
| GAAGTCGATGTTTGGCCTTGGCTTGTGAATATGACTGCGGATGTCATCTCACATACTGCTTTTG | |
| GAAGTAGCTATAAAGAAGGGCAGAGAATATTTCAACTACAAGGGGAATTGGCTGAGCTTATCGC | |
| ACAAGCTTTTAAGAAATCTTACATCCCTGGATCGAGGTTTTACCCAACAAAGAGCAATAGAAGG | |
| ATGAAAGCAATAGATAGAGAAGTAGACGTAATATTGAGAGGTATTGTGAGCAAACGGGAGAAAG | |
| CGAGAGAAGCTGGAGAACCAGCAAACGATGATTTGTTGGGGATATTGCTTGAATCCAATTCAGA | |
| GGAATCTCAAGGAAATGGAATGAGCGTAGAAGATGTGATGAAAGAGTGCAAGTTGTTTTATTTC | |
| GCGGGACAAGAGACAACTTCAGTACTTTTGGTGTGGACTATGGTTTTATTAAGCCATCACCAAG | |
| ACTGGCAAGCTCGCGCACGAGAGGAAGTGATGCAAGTACTCGGTGAAAATAATAAACCTGATAT | |
| GGAGTCCCTTAACAACCTTAAAGTCATGACTATGATCTTCAATGAGGTTTTGAGGCTATACCCT | |
| CCGGTGGCTCAGCTTAAACGAGTTGTCAACAAAGAAATGAAGCTCGGAGAGTTGACCCTTCCAG | |
| CTGGAATTCAAATTTACTTACCAACTATTCTTGTCCAGCGTGACACCGAGCTTTGGGGCGATGA | |
| TGCAGCGGATTTTAAACCCGAGCGGTTCAGAGACGGGCTCTCAAAGGCAACAAAGAACCAGGTC | |
| TCTTTCTTCCCCTTTGGATGGGGACCTAGGATTTGCATCGGTCAGAATTTTGCTATGTTGGAGG | |
| CAAAGATGGCAATGGCTTTGATTCTACAAAAGTTCTCCTTCGAGCTCTCTCCTTCTTATGTTCA | |
| CGCGCCTCAAACAGTCATGACCACTCGTCCCCAATTCGGAGCTCATCTAATTCTGCACAAGCTC | |
| TGA | |
| CYP72A14: (Ath29-3) | |
| DNA | |
| ATGGAGATATCAGTTTCTTCGGTAACATTTTCACTAGCTGTAGTTGTTGTGTCTTGGTGGGTAT | SEQ ID NQ:4 |
| GGAGAACGTTAAAGTGGGTTTGGTTCACACCAAAGATGCTTGAGCGTTCCCTGAGAAGACAAGG | |
| TCTTTCCGGAACTTCTTACACGCCTCTAATCGGCGATTTTAAAAAGATGATCAGCATGTTCATT | |
| GAGGCAACATCCAAACCCATCAAACCAACAGATGATATCACCCCTCGTGTCATGCCTCATCCCT | |
| TGCAAATGCTCAAGACTCATGGAAGGACTAACTTAACATGGTTTGGACCAATACCAACAATCAC | |
| CATAATGGATCCTGAGCAAATCAAGGAAGTGTTCAACAAAGTCTATGACTTCCAGAAGGCGCAT | |
| ACGTTTCCTTTAAGCAAAATACTAGGCACGGGACTCGTTAGTTATGATGGCGATAAATGGGCGC | |
| AACACCGAAGAATCATCAATCCGGCTTTCCACCTTGAGAAGATCAAGAATATGGTACATGTGTT | |
| CCACGAAAGCTGCAGCGAGCTTGTTGGTGAGTGGGACAAGTTAGTCTCGGATAAAGGGTCCTCA | |
| TGTGAGGTGGACGTGTGGCCTGGGCTTACGAGTATGACTGCAGATGTGATCTCCCGTACTGCTT | |
| TTGGTAGCAGCTACAGAGAAGGACACAGGATATTTGAACTCCAGGCAGAACTAGCACAGCTAGT | |
| CATGCAAGCTTTTCAGAAATTTTTTATTCCCGGATATATTTATCTCCCAACAAAGGGTAATAGA | |
| AGGATGAAAACAGCAGCCAGAGAAATCCAAGATATACTGAGAGGGATCATTAACAAAAGGGAAA | |
| GGGCAAGAGAATCTGGAGAAGCACCAAGCGAGGATTTGCTAGGTATACTTCTTGAATCAAACTT | |
| GGGGCAAACGGAAGGGAATGGAATGAGTACCGAGGATATGATGGAAGAATGCAAGTTGTTCTAT | |
| TTGGCCGGGCAAGAGACAACATCAGTACTTCTGGTTTGGACAATGGTTCTGTTGAGCCAACACC | |
| AAGATTGGCAGGCTCGTGCACGAGAGGAAGTGAAGCAAGTTTTTGGCGATAAACAACCTGATAC | |
| AGAAGGCCTTAACCAACTCAAAGTTATGACGATGATATTATATGAGGTCCTTAGGCTTTATCCT | |
| CCTGTAGTCCAGCTGACCCGAGCCATTCACAAAGAGATGAAGCTCGGAGATCTGACTTTACCAG | |
| GCGGTGTTCAGATCAGTCTACCTGTTCTGCTTGTCCATCGCGACACGGAGCTGTGGGGAAACGA | |
| TGCAGGGGAGTTCAAGCCTGAGAGATTCAAAGACGGCCTCTCAAAAGCAACAAAGAACCAAGTC | |
| TCCTTCTTTCCCTTTGCGTGGGGACCAAGGATCTGCATTGGCCAGAATTTTACATTGCTTGAGG | |
| CAAAGATGGCAATGAGTTTGATTCTACAGAGATTCTCCTTCGAGCTTTCTCCTTCCTATGTTCA | |
| CGCGCCTTACACAATCATCACCCTTTACCCACAGTTCGGAGCTCATCTTATGCTGCACAAGCTC | |
| TAA | |
| Sequence-translation of Ath30-2 | |
| Protein | |
| MSFSVVAALPVLVAVVVLWTWRIVKWVWIKPKMLESSLKRQGLTGTPYTPLVGDIKRNVD | SEQ ID NO:5 |
| MMMEARSKPINVTDDITPRLLPLALKMLNSHGKTFFIWIGPLPTIVITNPEQIKEVFNKV | |
| NDFEKASTFPLIRLLAGGLASYKGDKWASHRRIINPAFHLEKIKNMIPAFYHCCSEVVCQ | |
| WEKLFTDKESPLEVDVWPWLVNMTADVISHTAFGSSYKEGQRIFQLQGELAELIAQAFKK | |
| SYIPGSRFYPTKSNRRMKAIDREVDVILRGIVSKREKAREAGEPANDDLLGILLESNSEE | |
| SQGNGMSVEDVMKECKLFYFAGQETTSVLLVWTMVLLSHHQDWQARRAEEVMQVLGENNK | |
| PDMESLNNLKVMTMIFNEVLRLYPPVAQLKRVVNKEMKLGELTLPAGIQIYLPTILVQRD | |
| TELWGDDAADFKPERFRDGLSKATKNQVSFFPFGWGPRICIGQNFAMLEAKMAMALILQK | |
| FSFELSPSYVHAPQTVMTTRPQFGAHLILHKL- | |
| Sequence-translation of Ath29-3 | |
| Protein | |
| MEISVSSVTFSLAVVVVSWWVWRTLKWVWFTPKMLERSLRRQGLSGTSYTPLIGDFKKMI | SEQ ID NO:6 |
| SMFIEATSKPIKPTDDITPRVMPHPLQMLKTHGRTNLTWFGPIPTITIMDPEQIKEVFNK | |
| VYDFQKAHTFPLSKILGTGLVSYDGDKWAQHRRIINPAFHLEKIKNMVHVFHESCSELVG | |
| EWDKLVSDKGSSCEVDVWPGLTSMTADVISRTAFGSSYREGHRIFELQAELAQLVMQAFQ | |
| KFFIPGYIYLPTKGNRRMKTAAREIQDILRGIINKRERARESGEAPSEDLLGILLESNLG | |
| QTEGNGMSTEDMMEECKLFYLAGQETTSVLLVWTMVLLSQHQDWQARAREEVKQVFGDKQ | |
| PDTEGLNQLKVMTMILYEVLRLYPPVVQLTRAIHKEMKLGDLTLPGGVQISLPVLLVHRD | |
| TELWGNDAGEFKPERFKDGLSKATKNQVSFFPFAWGPRICIGQNFTLLEAKMAMSLILQR | |
| FSFELSPSYVHAPYTIITLYPQFGAHLMLHKL | |

| | |
|---|---|
| Synthetic DNA | |
| 5′ CGTTGGAGGATGGTGCATAG 3′ | SEQ ID NO:7 |
| Synthetic DNA | |
| 5′ GCTCGGTGGAATCAAATATCC 3′ | SEQ ID NO:8 |
| Synthetic DNA | |
| 5′ GATTCCTGACCCAGAGTTAG 3′ | SEQ ID NO:9 |
| Synthetic DNA | |
| 5′ GCAAGGAGAACTAGCTGAAC 3′ | SEQ ID NO:10 |
| Synthetic DNA | |
| 5′ CCGGATTATGATAGCTTGAG 3′ | SEQ ID NO:11 |
| Synthetic DNA | |
| 5′ CCTGTGCTTTGCCCATT 3′ | SEQ ID NO:12 |
| Synthetic DNA | |
| 5′ CGCAACCAGGCATCAG 3′ | SEQ ID NO:13 |
| Synthetic DNA | |
| 5′ ATGGATCCATGCTGCGGGAAGTCTCTC 3′ | SEQ ID NO:14 |
| Synthetic DNA | |
| 5′ ACGATATCGTCCGGTAGCAGCATAGAAG 3′ | SEQ ID NO:15 |
| Synthetic DNA | |
| 5′ ATCAGGATCCGTGAGAATCGGAGATGTC 3′ | SEQ ID NO:16 |
| Synthetic DNA | |
| 5′ TCGTGGTACCAAATCAGAGCTTGTGCAG 3′ | SEQ ID NO:17 |
| Synthetic DNA | |
| 5′ GTGGTACCATGGAGATATCAGTTTCTTCG 3′ | SEQ ID NO:18 |
| Synthetic DNA | |
| 5′CTGAATTCATGTGATTAGAGCTTGTGCAG 3′ | SEQ ID NO:19 |
| Synthetic DNA | |
| 5′ACGTTAACATGCTGCGGGAAGTCTCTC 3′ | SEQ ID NO:20 |
| Synthetic DNA | |
| 5′ACTCTAGACCGGTAGCAGCATAGAAGGA 3′ | SEQ ID NO:21 |
| Conserved motif (protein) | |
| EVLRLY | SEQ ID NO:22 |
| Conserved motif (protein) | |
| DVIS [KRH]XAFG | SEQ ID NO:23 |
| Conserved motif (protein) | |
| D[LI]LG[LIV][LM][VL][EQK] | SEQ ID NO:24 |

1. Isolated and purified deoxyribonucleic acid (DNA), that encodes a protein having ABA hydroxylase activity, characterized in that said DNA comprises SEQ ID NO:2, or a sequence that encodes a protein having at least 50% identity to said protein encoded by SEQ ID NO:2.

2. A protein exhibiting ABA hydroxylase activity, characterized in that said protein is encoded by SEQ ID NO:2 or has at least 50% identity to the protein encoded by SEQ ID NO:2.

3. A protein according to claim 2, characterized in that said protein has a heme binding domain and has a conserved amino acid sequence comprising SEQ ID NO:22 about 80 amino acids N-terminal to said heme binding domain.

4. A protein according to claim 3, characterized in that said protein has a conserved amino acid sequence comprising SEQ ID NO:23 located about 240 to 250 amino acids N-terminal to said heme binding domain.

5. A vector for transformation of plant cells, characterized in that said vector contains a protein-encoding deoxyribonucleic acid (DNA) sequence according to SEQ ID NO:2 or a DNA sequence that encodes a protein having at least 50% identity to the protein encoded by SEQ ID NO:2.

6. A plant having a genome, characterized in that the genome includes an introduced protein-encoding nucleotide sequence of SEQ ID NO:2, or an introduced DNA sequence that encodes a protein having at least 50% identity to the protein encoded by SEQ ID NO:2.

7. Isolated and purified deoxyribonucleic acid (DNA), that encodes a protein having ABA hydroxylase activity, characterized in that said DNA comprises SEQ ID NO:3, or a sequence that encodes a protein having at least 50% identity to said protein encoded by SEQ ID NO:3.

8. A protein exhibiting ABA hydroxylase activity, characterized in that said protein is encoded by SEQ ID NO:3 or has at least 50% identity to the protein encoded by SEQ ID NO:3.

9. A protein according to claim 8, characterized in that the protein has a heme binding domain and a conserved amino acid sequence of SEQ ID NO:22 about 80 amino acids N-terminal to the heme binding domain.

10. A protein according to claim 9, characterized in that the protein comprises SEQ ID NO:23 located about 240 to 250 amino acids N-terminal to the heme binding domain.

11. A vector for transformation of plant cells, characterized in that said vector contains a protein-encoding deoxyribonucleic acid (DNA) sequence according to SEQ ID NO:3 or a DNA sequence that encodes a protein having at least 50% identity to the protein encoded by SEQ ID NO:3.

12. A plant having a genome, characterized in that the genome includes an introduced protein-encoding nucleotide sequence of SEQ ID NO:3, or an introduced DNA sequence that encodes a protein having at least 50% identity to the protein encoded by SEQ ID NO:3.

13. Isolated and purified deoxyribonucleic acid (DNA), that encodes a protein having ABA hydroxylase activity, characterized in that said DNA comprises SEQ ID NO:4, or a sequence that encodes a protein having at least 50% identity to said protein encoded by SEQ ID NO:4.

14. A protein exhibiting ABA hydroxylase activity, characterized in that said protein is encoded by SEQ ID NO:4 or has at least 50% identity to the protein encoded by SEQ ID NO:4.

15. A protein according to claim 14, characterized in that said protein has a heme binding domain and has a conserved amino acid sequence comprising SEQ ID NO:22 about 80 amino acids N-terminal to said heme binding domain.

16. A protein according to claim 15, characterized in that said protein has a conserved amino acid sequence comprising SEQ ID NO:23 located about 240 to 250 amino acids N-terminal to said heme binding domain.

17. A vector for transformation of plant cells, characterized in that said vector contains a protein-encoding deoxyribonucleic acid (DNA) sequence according to SEQ ID NO:4 or a DNA sequence that encodes a protein having at least 50% identity to the protein encoded by SEQ ID NO:4.

18. A plant having a genome, characterized in that the genome includes an introduced protein-encoding nucleotide sequence of SEQ ID NO:4, or an introduced DNA sequence that encodes a protein having at least 50% identity to the protein encoded by SEQ ID NO:4.

19. A method of producing a transgenic plant comprising the step of introducing a nucleotide sequence into a genome of the plant or part thereof, and carrying out plant growth and development, characterized in that said nucleotide sequence is SEQ ID NO:2 or a DNA sequence that encodes a protein with at least 50% identity to the protein encoded by SEQ ID NO:2.

20. An isolated polynucleotide comprising a nucleotide sequence encoding a polypeptide selected from plant CYP72A P450 monooxygenases, characterized in that said plant CYP72A P450 monooxygenase has ABA hydroxylase activity.

21. An isolated polynucleotide according to claim 20, characterized in that the isolated polynucleotide is DNA.

22. An isolated polynucleotide according to claim 20, characterized in that the isolated polynucleotide is RNA.

23. An isolated polynucleotide according to claim 20, characterized in that said nucleotide sequence encodes an enzyme with ABA 7′-hydroxylase activity.

24. An isolated polynucleotide according to claim 20, characterized in that said nucleotide sequence encodes an enzyme with ABA 8′-hydroxylase activity.

25. An isolated polynucleotide corresponding to the complement of SEQ ID NO:1 or the complement of SEQ ID NO:2.

26. A chimeric gene comprising a deoxyribonucleic acid (DNA) operably linked to a regulatory element, characterized in that said DNA comprises sequence SEQ ID NO:2 or a DNA sequence that encodes a protein with at least 50% identity to the protein encoded by SEQ ID NO:2.

27. A chimeric gene comprising a deoxyribonucleic acid (DNA) operably linked to a regulatory element, characterized in that said DNA comprises sequence SEQ ID NO:3 or a DNA sequence that encodes a protein with at least 50% identity to the protein encoded by SEQ ID NO:3.

28. A chimeric gene comprising a deoxyribonucleic acid (DNA) operably linked to a regulatory element, characterized in that said DNA comprises sequence SEQ ID NO:4 or a DNA sequence that encodes a protein with at least 50% identity to the protein encoded by SEQ ID NO:4.

29. The chimeric gene of any one of claims 26 to 28, characterized in that said regulatory element is selected from constitutive, tissue selective and inducible promoters.

30. The chimeric gene of any one of claims 26 to 28, characterized in that said deoxyribonucleic acid is in an antisense orientation relative to the regulatory element.

31. The chimeric gene of any one of claims 26 to 28, characterized in that said deoxyribonucleic acid is in a sense orientation relative to the regulatory element.

32. A method of modifying catabolism of ABA in a plant, comprising: (a) introducing into a plant cell capable of being transformed and regenerated to a whole plant a genetic construct comprising a DNA expression cassette that comprises, in addition to the DNA sequences required for transformation and selection in plant cells, a DNA sequence that comprises SEQ ID NO:2 or a DNA sequence that encodes a protein with at least 50% identity to the protein encoded by SEQ ID NO:2, operably linked to a transcriptional regulatory region, and (b) recovery of a plant which contains said additional deoxyribonucleic acid.

33. A method of modifying catabolism of ABA in a plant, comprising: (a) introducing into a plant cell capable of being transformed and regenerated to a whole plant a genetic construct comprising a DNA expression cassette that comprises, in addition to the DNA sequences required for transformation and selection in plant cells, a DNA sequence that comprises SEQ ID NO:3 or a DNA sequence that encodes a protein with at least 50% identity to the protein encoded by SEQ ID NO:3, operably linked to a transcriptional regulatory region, and (b) recovery of a plant which contains said additional deoxyribonucleic acid.

34. A method of modifying catabolism of ABA in a plant, comprising: (a) introducing into a plant cell capable of being transformed and regenerated to a whole plant a genetic construct comprising a DNA expression cassette that comprises, in addition to the DNA sequences required for transformation and selection in plant cells, a DNA sequence that comprises SEQ ID NO:4 or a DNA sequence that encodes a protein with at least 50% identity to the protein encoded by SEQ ID NO:4, operably linked to a transcriptional regulatory region, and (b) recovery of a plant which contains said additional deoxyribonucleic acid.

35. A method of modifying the catabolism of ABA analogs in a plant comprising: (a) introducing into a plant cell capable of being transformed and regenerated to a whole plant a genetic construct comprising a DNA expression cassette that comprises, in addition to the DNA sequences required for transformation and selection in plant cells, a DNA sequence that comprises SEQ ID NO:4 or a DNA sequence that encodes a protein with at least 50% identity to the protein encoded by SEQ ID NO:4, operably linked to a transcriptional regulatory region; (b) recovery of a plant which contains said additional DNA; and (c) treating the plant with an ABA analog to recover a plant that has a novel physiological response to said analog, when compared to a non-recombinant plant treated with said ABA analog.

36. A method according to any one of claims 32 to 35, characterized in that the plant is a dicotyledonous plant.

37. A method according to any one of claims 32 to 35, characterized in that the plant is a monocotyledonous plant.

38. Isolated and purified deoxyribonucleic acid (DNA), characterized in that said DNA includes SEQ ID NO:1, or a DNA that encodes a protein with at least 50% identity to the protein encoded by SEQ ID NO:1.

39. An expression cassette comprising DNA, characterized in that said DNA comprises SEQ ID NO:2 or a sequence that encodes a protein with at least 50% identity to the protein encoded by SEQ ID NO:2, operably linked to a regulatory element.

40. An expression cassette comprising DNA, characterized in that said DNA comprises SEQ ID NO:3 or a sequence that encodes a protein with at least 50% identity to the protein encoded by SEQ ID NO:3, operably linked to a regulatory element.

41. An expression cassette comprising DNA, characterized in that said DNA comprises SEQ ID NO:4 or a sequence that encodes a protein with at least 50% identity to the protein encoded by SEQ ID NO:4, operably linked to a regulatory element.
